SEQUENCE LISTING (3 sequences)

SEQ ID NO: 1 - protein, 13 aa, Artificial Sequence
  Synthetic
HSLGKWLGHPDKF

SEQ ID NO: 2 - DNA, 30 bp, Artificial Sequence
  Synthetic
atcggatcctgtggacacatcgagatttca

SEQ ID NO: 3 - DNA, 34 bp, Artificial Sequence
  Synthetic
atcggatccgtcagatggatcatgggtcctcagg

(19) United States
(12) Patent Application Publication
(10) Pub. No.: US 2011/0117092 A1
(43) Pub. Date: May 19, 2011

(54) COMPOSITIONS AND METHODS FOR INHIBITING G-CSFR

(75) Inventors: Benjamin M. Segal, Ann Arbor, MI (US); Praveen Rao, Ann Arbor, MI (US)

(73) Assignee: THE REGENTS OF THE UNIVERSITY OF MICHIGAN, Ann Arbor, MI (US)

(21) Appl. No.: 12/906,548

(22) Filed: Oct. 18, 2010

Related U.S. Application Data
(60) Provisional application No. 61/253,138, filed on Oct. 20, 2009.

(51) Int. Cl. A61K 39/395; C07K 16/24; C07H 21/02; C07H 21/00; A61K 31/713; A61K 31/7088; A61P 29/00; A61P 25/28; A61P 25/00; C07K 16/28

(52) U.S. Cl. 424/134.1; 530/387.3; 530/389.2; 536/24.5; 424/158.1; 514/44 A; 530/389.6; 424/173.1

(57) ABSTRACT

The present invention relates to therapeutic targets for multiple sclerosis and other inflammatory and neurological diseases. In particular, the present invention relates to altering G-CSF/G-CSFR signaling in the treatment of such disorders.

### CROSS REFERENCE TO RELATED APPLICATIONS

This application claims priority to U.S. Provisional Patent Application Ser. No. 61/253,138, filed Oct. 20, 2009, hereby incorporated by reference in its entirety.

### FIELD OF THE INVENTION

The present invention relates to therapeutic targets for multiple sclerosis and other inflammatory and neurological diseases. In particular, the present invention relates to altering G-CSF/G-CSFR signaling in the treatment of such disorders.

### BACKGROUND OF THE INVENTION

Multiple sclerosis (MS) is the most common non-traumatic cause of neurological disability among young adults in Western nations. It currently afflicts 400,000 individuals in the United States and more than 1,000,000 individuals worldwide. Over the past two decades the United States Food and Drug Administration (FDA) has approved five disease modifying therapies for the long term treatment of individuals with RRMS. These therapies include three different formulations of recombinant interferon β and glatiramer acetate. Double-blind, placebo-controlled, randomized trials have demonstrated that each of the formulations of interferon β as well as glatiramer acetate reduce the frequency of clinical exacerbations by approximately 30% and significantly suppress the incidence of gadolinium enhancing lesions detected by serial magnetic resonance imaging. Furthermore, retrospective epidemiological studies indicate that maintenance therapy with interferon β slows the rate of accumulation of disability in RRMS. Despite these accomplishments in MS therapeutics, the currently available therapies are only modestly effective. None are curative and most patients continue to transition into secondary progression despite treatment. More aggressive approaches with globally immunosuppressive chemotherapies are beleaguered by serious adverse side effects, including an increased risk of neoplasia and infection and, in the case of mitoxantrone, cardiotoxicity.

Thus, development of new therapeutic targets and agents is needed.

### SUMMARY OF THE INVENTION

The present invention relates to therapeutic targets for multiple sclerosis and other inflammatory and neurological diseases. In particular, the present invention relates to regulating granulocyte colony stimulating factor (G-CSF) and/or its receptor, granulocyte colony stimulating factor receptor (G-CSFR) in the treatment of such disorders.

For example, in some embodiments, the present invention provides a pharmaceutical composition comprising a pharmaceutical agent (e.g., a G-CSFR FC-fusion, an anti-G-CSF antibody, an anti-G-CSF receptor antibody, a small molecule, an siRNA that inhibits the expression of G-CSF or G-CSFR or an antisense nucleic acid that inhibits the expression of G-CSF or G-CSFR or that inhibits at least one activity of a G-CSF or G-CSFR protein). In some embodiments, the composition reduces or eliminates symptoms or prevents relapses of a neuroinflammatory condition (e.g., MS).

In further embodiments, the present invention provides a method of treating a neuroinflammatory condition, comprising: administering a composition that inhibits at least one activity of a G-CSF or G-CSFR protein to a subject diagnosed with a neuroinflammatory condition (e.g., MS) under conditions such that symptoms of the neuroinflammatory condition are reduced or eliminated. In some embodiments, the composition is a G-CSFR FC-fusion, an anti-G-CSF antibody, an anti-G-CSF receptor antibody, a small molecule, an siRNA that inhibits the expression of G-CSF or G-CSFR or an antisense nucleic acid that inhibits the expression of G-CSF or G-CSFR or that inhibits at least one activity of a G-CSF or G-CSFR protein. In some embodiments, the composition accelerates recovery of the subject from symptoms of the neuroinflammatory condition or prevents relapses of symptoms of the neuroinflammatory condition.

Additional embodiments are described herein.

### DESCRIPTION OF THE FIGURES

FIG. 1. CD11c+ cells are present in EAE infiltrates, form clusters with infiltrating T cells and express markers indicative of myeloid DC. (A) C57BL/6 mice (n=10) were sacrificed on day 12 following immunization with MOG35-55, in the midst of the 1st episode of clinical EAE (mean score of 3.5). (B) In an independent experiment the vast majority of spinal cord CD11c+ cells were further characterized as CD8α−, indicative of myeloid dendritic cells. (C) Shown is a frozen section of a spinal cord from a representative C57BL/6 mouse with EAE.

FIG. 2. MOG-specific T cells produce IFNγ and IL-17 upon activation with CNS APC. (A) Purified CD45RBhi 2D2 cells (naive T cell receptor transgenic CD4+ T cells specific for MOG) were cultured with each of those APC subpopulations or syngeneic bone marrow derived dendritic cells (BMDCs, used as a positive control) at a 1:4 ratio. (B) CD4+ T cells were isolated from draining lymph nodes of MOG35-55 primed C57BL/6 mice and cocultured with APCs as described in (A) above.

FIG. 3. CD11b+CD11c+ cells isolated from mice with EAE produce IL-23 upon stimulation ex vivo. (A) CNS APCs were cultured with CpG containing or control ODN for 48 hours. The cells were harvested, washed ×3, and stained on the surface with fluorochrome-conjugated antibodies specific for CD11b and CD11c and intracellularly with anti-IL-12p40 prior to flow cytometric analysis. (B) MOG-specific CD4+ T cells were cocultured with each APC population indicated in the absence or presence of MOG35-55. IL-23 levels were measured at 72 hours by ELISA using IL-23p19-specific antibodies.

FIG. 4. CD11c+CD11b+ cells are recruited to the CNS from the periphery during EAE. (A) Bone marrow chimeras were constructed by injecting CD45.1+ congenic bone marrow cells into lethally irradiated CD45.2+C57BL/6 hosts. (B) Reconstituted chimeric mice were immunized with MOG35-55 in CFA to induce EAE.

FIG. 5. Myeloid precursor cells expand in the blood prior to clinical episodes of EAE and are contained within the CD11b+Ly-6Chi population. (A) SJL mice were immunized with PLP139-151 in CFA. The data shown are from one of 3 similar experiments. (B) C57/B6 mice were immunized with MOG/CFA. Six days later, peripheral blood cells were stained for CD11b and Ly-6C and sorted into Ly-6Chi, Ly-6Cint and Ly-6neg subsets. (C) Sorted cells were plated in methylcellulose cultures as described in (A) and GM-CFUs were counted on day 7. (D) H&E staining of sorted CD11b+Ly-6Chi cells. (E) CD115, CD62L and Ly6G levels were measured on gated CD11b+Ly-6Chi cells by flow cytometric analysis. Broken lines represent isotype controls. (B-E) Data are representative of at least two experiments.

FIG. 6. Ly-6C+ cells accumulate in the blood and CNS prior to onset of EAE. (A) PBMC from naive and PLP139-151-immunized SJL mice were analyzed by flow cytometry. Dot plots are gated on CD11b+CD115+ cells; percentages of each subset among total blood leukocytes are indicated. (B) Absolute numbers of circulating monocytes/ml of blood in naive and PLP139-151-immunized SJL mice (* p<0.05, ** p<0.02 by comparison to naive). (C) Ly-6C+ monocytes were sorted from immunized SJL mice and analyzed for MHCII and CD11c expression either immediately ex vivo or after a 48 hour culture with GM-CSF. Data shown are representative of 5 separate experiments. (D) Spinal cord mononuclear cells were harvested from naive and PLP139-151-immunized SJL mice and subjected to flow cytometric analysis. The lowest panels show CD11c and MHC Class II staining of CD11b+LyChi gated peripheral blood mononuclear cells from mice in the preclinical (left) or symptomatic (right) stages of EAE. The gates used are depicted in the middle panels. Data shown are representative of 4 separate experiments.

FIG. 7. Ly-6C+ monocytes migrate to the CNS during EAE and upregulate CD11c and MHCII. (A) Left, Expression of FITC and CD11b by CD115+ gated cells 5 days following liposome treatment and 4 days following FITC microsphere injection. Right, Ly-6C expression on FITC+CD115+ blood monocytes from clodronate vs. PBS liposome treated animals. Histograms are based on cells that fall within the R1 gate depicted in A. (B) Spinal cord mononuclear cells were harvested from clodronate (left) or PBS liposome (right) treated mice during peak EAE and analyzed for FITC expression. (C) CD11b+MHCII+Ly-6C+ cells were sorted from the blood and CNS of mice with EAE and analyzed by real-time RT-PCR for the genes shown. The data are shown as fold expression in CNS infiltrating cells over circulating cells. (A-C) All experiments shown were repeated three times with similar results

FIG. 8. CD11b+Ly6Chi blood monocytes migrate to the CNS following transfer into hosts with EAE. (A) Transferred CD45.1-CD11b+ donor cells appear in the R1 gate (left panel). (B) Donor cells within the R1 gate were analyzed for expression of the markers indicated. Data is representative of three independent experiments with similar results.

FIG. 9. Enrichment of Ly-6C+ cells in the circulating monocyte pool enhances EAE. (A) The absolute number of total monocytes and Ly6C+ monocytes/ml of blood in adoptive transfer recipients at serial time points following treatment with clodronate liposomes. (B) The absolute number of circulating Ly-6C+ monocytes/ml blood 5 days following treatment with either clodronate or PBS liposomes. (C) Clinical course of mice treated with a single dose of clodronate or PBS liposomes 18 hours after the adoptive transfer of encephalitogenic cells. Data shown represents the results from three separate experiments.

FIG. 10. GM-CSF triggers accelerated myelopoiesis during EAE. (A) Left, CD11b+CD115+Ly-6C+ or Ly-6C− blood cells were enumerated 7 days post-immunization of C57BL/6 WT and GM-CSF−/− mice with MOG peptide in CFA. The data are presented as the fold increase in each subset over their frequency in unimmunized counterparts. (B) Left, the number of circulating Ly-6C+ monocytes/ml of blood in anti-GM-CSF versus control antibody treated WT mice on day 7 post-immunization with MOG peptide. Right, clinical scores of MOG-immunized WT mice treated with either control antibody or anti-GM-CSF. (C) Left, Frequency of Ly6C+ blood monocytes on day 8 post active immunization of WT or GM-CSF−/− mice. Right, clinical scores of MOG-immunized WT and GM-CSF−/− mice. Some GM-CSF−/− mice received 5 μg of rmGM-CSF every day from days 0-16 post-immunization.

FIG. 11. GM-CSF receptor expression is not required for the accumulation of Ly6ChiCD11b+ cells in the blood or CNS of myelin immunized mice immediately before the clinical onset of EAE. The dot plots are gated on Ly6ChiCD11b+ cells among peripheral blood (upper dot plot) and CNS (lower dot plot) mononuclear cells pooled from five mice/group. Data shown are representative of three separate experiments.

FIG. 12. Polymorphonuclear cells (PMN) accumulate in the blood and infiltrate the CNS during the preclinical and active stages of EAE. (a-b) Peripheral blood leukocytes were analyzed by flow cytometry to determine the percentage of PMN (identified as Ly6G⁺, 7/4⁺, MHC class II⁻ cells). (a) Representative FACS profiles. (b) Percentages of peripheral blood leukocytes were averaged over 4 mice per group. * P<0.001 compared to naive. (c-d) Spinal cord-infiltrating cells were isolated from PLP₁₃₉₋₁₅₁-immunized SJL mice immediately prior to, or on the day of, clinical EAE onset. Cells were pooled from 10-20 mice per group for flow cytometric analysis. Spinal cord-infiltrating cells from mice immunized with NP₂₆₀₋₂₈₃ were used as controls. PMN were identified as in a. (c) Representative FACS profiles from each group. Plots are gated on MHC Class II⁻ cells. (d) Absolute number PMN/spinal cord and percent of CNS-infiltrating PMN. The data represent the mean±s.d. of 4 independent experiments. * P<0.05 compared to control. (e) Histologic sections of spinal cords from PLP₁₃₉₋₁₅₁- or NP₂₆₀₋₂₈₃-immunized mice were giemsa stained. PMN (arrows) were identified by their characteristic nuclear morphology (inset). Scale bar represents 50 μm. The sections shown are representative of 4 mice per group. (f) RNA was extracted from spinal cords between days 8-12 post immunization with PLP₁₃₉₋₁₅₁ (closed squares) or NP₂₆₀₋₂₈₃ (open circles) for analysis by real time RT-PCR. Data represent fold-induction relative to naïve spinal cords (n=5 mice per group)* P<0.05 PLP-compared to NP-immunized mice.

FIG. 13. PMN depletion prevents BBB disruption and clinical and histologic manifestations of EAE in PLP-immunized mice. SJL mice were injected with either RB6 (open circles) or control IgG (closed squares) (0.5 mg/dose) every other day from day 8 through day 16 (a-c, f-g) or day 21 through day 27 (d-e) post-immunization with PLP₁₃₉₋₁₅₁ in CFA. (a) Peripheral blood leukocytes were collected at serial time points and analyzed by flow cytometry. PMN are identified as CD11b⁺, 7/4⁺, MHC class II⁻ cells. The time course was generated by averaging the percent of PMN over 6 mice per group. A representative FACS profile, gated on MHC Class II⁻ cells, of each group is shown. (b) The mean daily clinical score of each group is shown. n=6 per group. (c) Mice were injected i.v. with Evans Blue dye during the time of the first episode of EAE in the IgG treated group. Spinal cords and kidneys were removed 2 hours later. Dye extravasation was assessed by spectrophotometry of tissue homogenate supernatants. Relative permeability is calculated as (μg E. blue/g spinal cord)/(μg E. blue/g kidney). Data represent mean±s.d. of 6 mice/group. * P<0.05 compared to naïve. (d) The mean daily clinical score of each group (n=8) is shown. (e) Spinal cords and kidneys were removed during the time of relapse in the IgG treated group, and analyzed as in c (n=6 per group). * P<0.05 compared to naïve. (f-g) Spinal cords were harvested and fixed on day 14 following immunization with PLP₁₃₉₋₁₅₁ and treatment with either control IgG (f) or RB6 (g) according to the schedule depicted in a. Sections were giemsa stained to visualize cell morphology. Scale bar represents 200 μm (f left, and g), and 50 μm (f right). Images are representative of sections from 4 mice per group.

FIG. 14. PLP-specific peripheral CD4⁺ responses are not altered by PMN depletion, but CNS upregulation of inflammatory markers is blocked. (a-b) CD4⁺ T cells were isolated from draining lymph nodes of PLP-immunized SJL mice that had been treated with either IgG (filled bars) or RB6 (open bars) during priming. The purified T cells were stimulated with naïve, T-depleted splenocytes and PLP₁₃₉₋₁₅₁ (25 μg/ml) and subjected to ELISPOT (a) and [³H]-thymidine uptake proliferation (b) assays. The ELISPOT data shown was generated by subtracting background spots that appeared in the absence of antigenic challenge (1-5/well). (c) Spinal cords from PLP-immunized mice that had been treated with either IgG or RB6 were analyzed by real time RT-PCR. Data represent fold-induction relative to naïve spinal cords (n=5 mice per group). * P<0.05 IgG-compared to RB6-treated mice. n.s. not significantly different from naive spinal cords.

FIG. 15. Expression of pro-inflammatory molecules is associated with clinical disease activity. SJL mice were immunized with PLP₁₃₉₋₁₅₁ (filled bars) or NP₂₆₀₋₂₈₃ (open bars) and RNA was isolated during distinct stages of relapsing-remitting EAE for real time RT-PCR analysis. Data represent fold-induction compared to naive spinal cords (n=4 per group). * P<0.05 PLP-immunized compared to NP-immunized mice. n.s. not significantly different from naive spinal cords.

FIG. 16. CXCR2^{-/-} mice are resistant to EAE and fail to develop CNS inflammatory infiltrates. BALB/c CXCR2^{+/+}, CXCR2^{+/-} and CXCR2^{-/-} mice were immunized with PLP₁₈₅₋₂₀₆ in CFA to induce EAE. (a) Spinal cords were removed on day 15 post-immunization and giemsa stained. Representative sections are shown from 6 mice per group. Scale bar represents 50 μm. PMN were detected in CXCR2^{+/-} cords (arrows). (b) The mean daily clinical score of each group is shown (+/+n=5, +/−n=8, −/−n=7). The experiment was repeated three times with similar results. (c-d) CD4⁺ T cells were purified from draining lymph nodes on day 10 post-immunization and stimulated in vitro with naive T-depleted splenocytes and 25 μg/ml PLP₁₈₅₋₂₀₆. Data are representative of 4 independent experiments. (c) [³H]-thymidine uptake was used as a measure of proliferation. (d) ELISPOT assays were performed to determine the frequency of PLP₁₈₅₋₂₀₆ specific cytokine producing cells. Background counts (without antigen) were subtracted to generate the data shown.

FIG. 17. Wildtype neutrophils restore disease susceptibility and CNS inflammation in CXCR2^{-/-} mice. BALB/c CXCR2^{+/+} and CXCR2^{-/-} mice were immunized with PLP₁₈₅₋₂₀₆. 5×10⁶ purified bone marrow PMN or BMMac were transferred into CXCR2^{-/-} mice daily between days 10 and 14 post-immunization. (a) Spinal cords were removed between days 13 and 15 post-immunization, fixed, and H+E stained. Representative sections of 3 mice per group are shown. Scale bars represent 200 μm (left four panels) and 50 μm (right two panels). (b) RNA isolated from spinal cords between days 13 and 15 post-immunization was analyzed by real time RT-PCR. Data represent fold-induction compared to naive spinal cords (n=4 per group). * P<0.03 compared to CXCR2^{-/-} mice. n.d. not detectable.

FIG. 18. The plasmids used to construct human and mouse G-CSFR-Fc fusion proteins.

FIG. 19. Western blot analysis of murine G-CSFR. Western blot analysis of supernatants (lanes 1-3) and extracts (4-6) of untransfected HEK293T cells (1, 4) or of HEK293 T cells transfected with empty vector (2, 5) or with the plasmid containing the extracellular domain of MAdCAM-1-Fc (3, 6).

FIG. 20. Administration of murine G-CSFR-Fc fusion protein following the onset of clinical EAE is therapeutic. SJL mice were immunized s.c. with 100 μg proteolipid protein peptide 139-151 (PLP₁₃₉₋₁₅₁, HSLGKWLGHPDKF) emulsifed in CFA. One day following EAE onset (day 12) the mice were matched for clinical scores and divided into two groups (n=10/group). Each group received either G-CSFR-Fc or a control Fc by i.p. injection on days 12, 14, 16 and 18 post immunization (0.75 mg/mouse/injection). * p<0.05

FIG. 21. Treatment of myelin-immunized mice with impaired gait with murine G-CSFR-Fc accelerates recovery. SJL mice were immunized s.c. with 100 μg proteolipid protein peptide 139-151 (PLP₁₃₉₋₁₅₁, HSLGKWLGHPDKF) emulsified in CFA. On day 12 post immunization (average clinical score 2.5) mice were matched for clinical scores and divided into two groups (n=7/group). Each group received either G-CSFR-Fc or a control Fc by i.p. injection on days 12, 14, 16, 18 and 20 post immunization (0.75 mg/mouse/injection). * p<0.05

FIG. 22. Treatment of myelin-immunized mice with human G-CSF-R-Fc inhibits clinical EAE. EAE was induced in SJL mice as described in FIGS. 20 and 21. Human G-CSFR-Fc or a control human Fc was injected i.p. on days 3, 6, 9 and 12 post immunization (n=5/group). The experiment shown is representative of two. * p<0.05

FIG. 23. IL-12 and IL-23 modulated T cells induce distinct panels of chemokines and effector molecules in the CNS. SJL mice were injected with IL-12 or IL-23 polarized PLP-reactive CD4+ T cells. Real time RT-PCR was performed using RNA isolated from individual spinal cords at peak disease. Levels of ELR⁺ CXC chemokines (A), ELR⁻ CXC chemokines (B) and G-CSF and NOS2 (C) were normalized to GAPDH and averaged over 4-6 mice per group. The data shown is representative of three independent experiments. (** p<0.001; * p<0.05 by comparison to recipients of IL-12 modulated T cells).

FIG. 24. G-CSFR-Fc inhibits G-CSF driven expansion of NSF-60 cells. NSF-60 cells were incubated for 5 days in tissue culture media (5×10⁵ cells in 10 ml) with recombinant M-CSF (62 ng/ml), recombinant G-CSF (0.125 ng/ml) and either control Fc protein (15 μg/ml) or G-CSFR-Fc fusion protein (15 μg/ml). In some cases, cells cultured in the presence of G-CSFR-Fc were washed on day 4 and recultured in the presence of recombinant G-CSF alone for the last 24 hours. At day 5, cells were harvested and counted under a light microscope by trypan blue exclusion.

### DEFINITIONS

To facilitate an understanding of the present invention, a number of terms and phrases are defined below:

As used herein, the term “neuroinflammatory condition” refers to any condition (e.g., injury) or disease that results in inflammation of a neurological tissue. Examples include, but are not limited to, multiple sclerosis (MS), acute trauma (e.g., head injury or spinal cord injury), Alzheimer's disease, amyotrophic lateral sclerosis (ALS, also known as Lou Gehrig's disease), acute disseminated encephalomyelitis, Bell's palsy, diffuse sclerosis, neurosarcoidosis, CNS complications of collagen vascular diseases (including Sjogren's disease, polyarteritis nodosa, systemic lupus erythematosus, Wegener's granulomatosis), takayasu arteritis, temporal/giant cell arteritis, tolosa-hunt syndrome, primary CNS angiitis, transverse myelitis, Susac's syndrome, PANDAS (pediatric autoimmune neuropsychiatric disorders associated with streptococcal infections), sydenham's chorea, adrenomyeloneuropathy, Guillain-Barre syndrome, chronic inflammatory demyelinating polyneuropathy, polymyositis, neurobehcet's disease, paraneoplastic syndromes, limbic encephalitis, Lambert-Eaton myasthenic syndrome and myasthenia gravis.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “non-human animals” refers to all non-human animals including, but are not limited to, vertebrates such as rodents, non-human primates, ovines, bovines, ruminants, lagomorphs, porcines, caprines, equines, canines, felines, ayes, etc.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment is retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tm of the formed hybrid, and the G:C ratio within the nucleic acids. A single molecule that contains pairing of complementary nucleic acids within its structure is said to be “self-hybridized.”

As used herein, the term “Tm” is used in reference to the “melting temperature.” The melting temperature is the temperature at which a population of double-stranded nucleic acid molecules becomes half dissociated into single strands. The equation for calculating the Tm of nucleic acids is well known in the art. As indicated by standard references, a simple estimate of the Tm value may be calculated by the equation: Tm=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tm.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/lNaH2PO4H2O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/lNaH2PO4H2O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH2PO4H2O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

As used herein, the term “siRNAs” refers to small interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to, or substantially complementary to, a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “epitope” as used herein refers to that portion of an antigen that makes contact with a particular antibody.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., neuroinflammatory disease). Test compounds comprise both known and potential therapeutic compounds. A test compound can be determined to be therapeutic by screening using the screening methods of the present invention. In some embodiments of the present invention, test compounds include antisense compounds.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

### DETAILED DESCRIPTION OF THE INVENTION

The present invention relates to therapeutic targets for multiple sclerosis and other inflammatory and neurological diseases. In particular, the present invention relates to altering G-CSF/G-CSFR signaling in the treatment of such disorders.

The present invention is not limited to a particular mechanism. Indeed, an understanding of the mechanism is not necessary to practice the present invention. Nonetheless, as is described below, it is contemplated that G-CSF contributes to disease pathogenesis by one or more of several mechanisms including: (i) enhancing the mobilization of precursor CD11b+ cells that give rise to mature myeloid cells that infiltrate the target organ; and (ii) initiating blood-target organ breakdown via activation of neutrophils at the site of lesion formation.

It is well established that the disease process in experimental autoimmune encephalomyelitis (EAE) is initiated by the activation of myelin-specific CD4+ T cells (Pettinelli and McFarlin, J Immunol 127, 1420-1423 (1981)). These cells migrate to the CNS, undergo reactivation in situ and subsequently recruit a large population of non-specific leukocytes to developing lesions (Zamvil and Steinman, Annu Revlmmunol 8, 579-621 (1990)). CD11b+ myeloid cells are a major constituent of established inflammatory infiltrates in EAE and multiple sclerosis (MS). Several lines of evidence indicate that they play a critical role within the central nervous system during the effector phase. First, they serve as local antigen presenting cells (APCs) for the reactivation of myelin-specific memory cells during relapses and/or progression as well as for the activation of naïve autoreactive T cells in the context of epitope spreading (Greter et al., Nat Med 11, 328-334 (2005); Katz-Levy et al., J Clin Invest 104, 599-610 (1999); McMahon et al., Nat Med 11, 335-339 (2005)). Second they secrete IL-12p40 monokines that help to promote and shape the local neuroinflammatory response (Issazadeh et al., J Neuroimmunol 61, 205-212 (1995); Segal et al., J Exp Med 187, 537-546 (1998); Windhagen et al., J Exp Med 182, 1985-1996 (1995)). Third, they directly inflict damage to myelin and axons via the release of toxic factors (such as free oxygen radicals, metalloproteinases and TNF) and by direct phagocytosis of the myelin sheath (Epstein et al., J Neurol Sci 61, 341-348 (1983); Raivich and Banati, BrainRes Brain Res Rev 46, 261-281 (2004)).

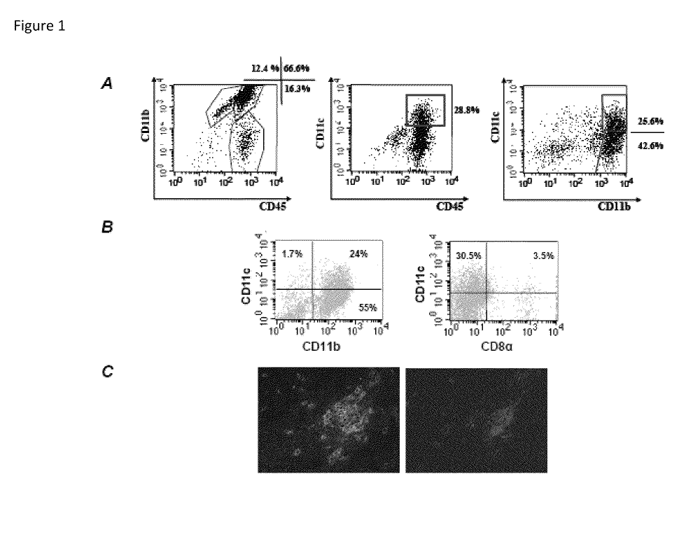

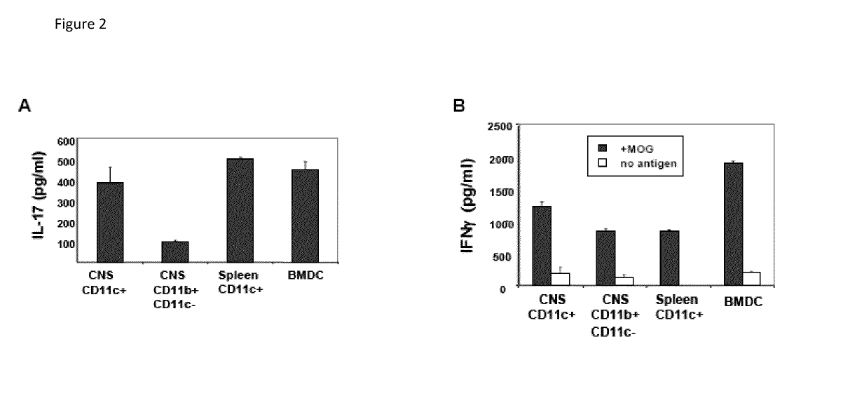

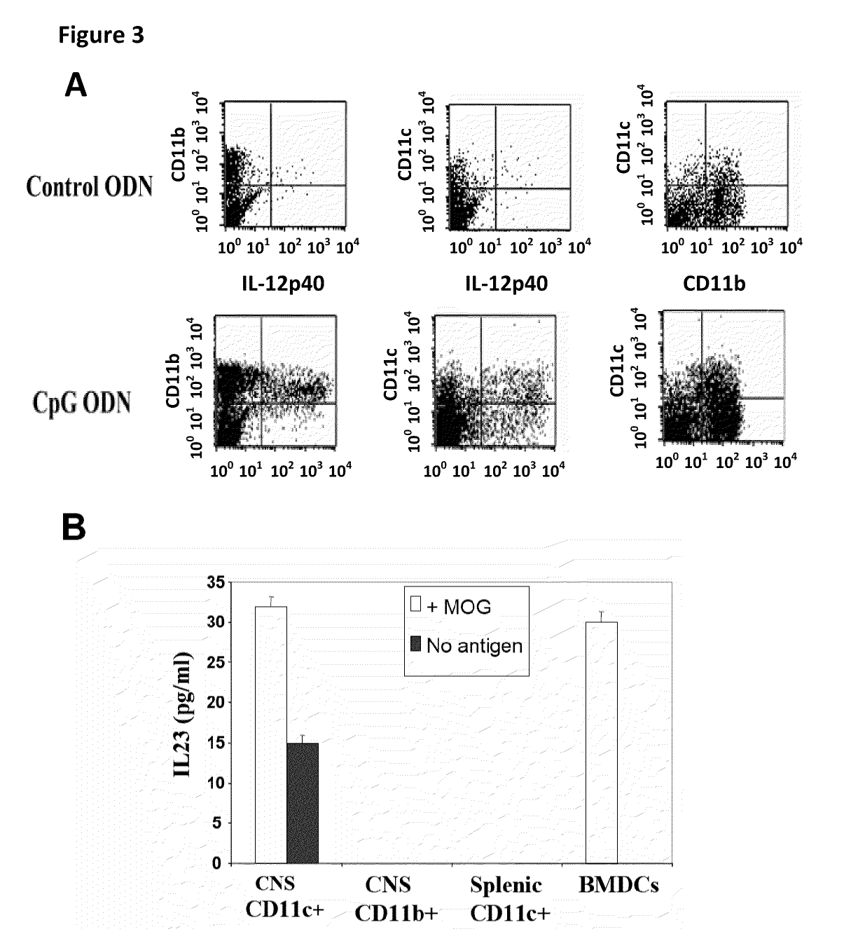

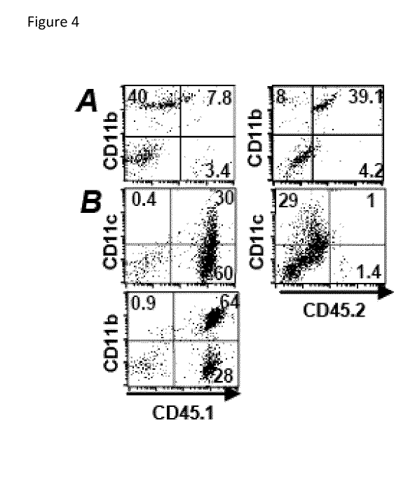

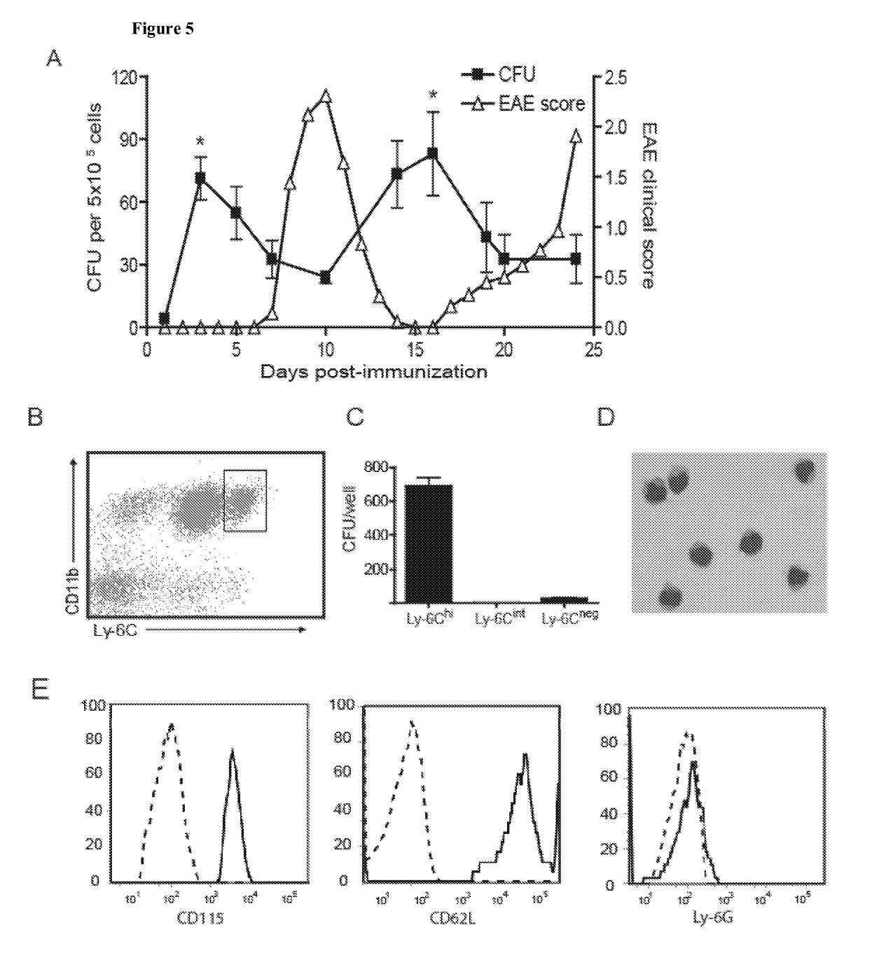

Myeloid cells in EAE lesions consist primarily of CD45hiCD11b+CD11c+ and CD45hiCD11b+CD11c− subsets (FIG. 1) (Deshpande et al., J Neuroimmunol 173, 35-44 (2006); Fischer and Reichmann, J Immunol 166, 2717-2726 (2001)). Both of these populations are capable of secreting IL-12p40 monokines and stimulating primed myelin-specific T cells to proliferate and secrete IFNγ and IL-17 in an antigen-specific manner (FIGS. 2, 3). The CD45hiCD11b+CD11c− population represents blood borne macrophages, since it has been firmly established that they can be distinguished from resident microglia by CD45 expression levels (the former being CD45hi and the latter, CD45int) (Ford et al., J Immunol 154, 4309-4321 (1995); Sedgwick et al., Proc Natl Acad Sci USA 88, 7438-7442 (1991)). Longitudinal histological studies have demonstrated that large numbers of macrophages invade the CNS from the circulation during the preclinical and acute stages of EAE (Moore et al., Lab Invest 57, 157-167 (1987)). Furthermore, depletion of circulating macrophages with systemic agents that leave parenchymal microglia intact prevents EAE (Tran et al., J Immunol 161, 3767-3775 (1998)). In vitro studies have demonstrated that CNS CD11c+ cells arise from microglia that transform into dendritic-like cells under the influence of proinflammatory stimuli and growth factors (Santambrogio et al., Developmental plasticity of CNS microglia. Proc Natl Acad Sci USA 98, 6295-6300 (2001)). However, experiments with bone marrow chimeric mice indicate that virtually all of the CD45hiCD11b+ myeloid cells in CNS tissues of mice with active EAE, including those within the CD11c+ subset, are derived from radiosensitive peripheral hematopoetic cells as opposed to radioresistant resident microglia (FIG. 4) (Greter et al., supra). It is contemplated that G-CSF drives the mobilization of bone marrow precursor cells into the circulation that ultimately give rise to CNS infiltrating CD11b+ cells during lesion formation and clinical exacerbations of neuroinflammatory disease, including MS.

Under steady-state conditions, mature myeloid cells are maintained within lymphoid and peripheral tissues through controlled release of bone marrow progenitors/precursors into the peripheral circulation. In the setting of infection or injury, myeloid cell mobilization is accelerated to meet the demands imposed by the increased turnover of macrophages and dendritic cells at the site of inflammation (Dunay et al., Immunity 29, 306-317 (2008); Nahrendorf et al., J Exp Med 204, 3037-3047 (2007)). The pathways underlying expansion of peripheral myeloid cell pools under stress serve an adaptive role by reinforcing host protection against infectious agents and by promoting wound healing (Dunay et al., supra). Conversely, leukocyte mobilizing pathways are subverted to sustain target organ inflammation during relapsing or chronic autoimmune disease. For example, the number of macrophages and dendritic cells in the CNS contracts during remissions and rebounds during exacerbations of EAE, indicating that myeloid precursors are released at a heightened rate prior to, or in concert with, clinical disease activity (Begolka et al., J Immunol 161, 4437-4446 (1998); Jee et al., J Neuroimmunol 128, 49-57 (2002)).

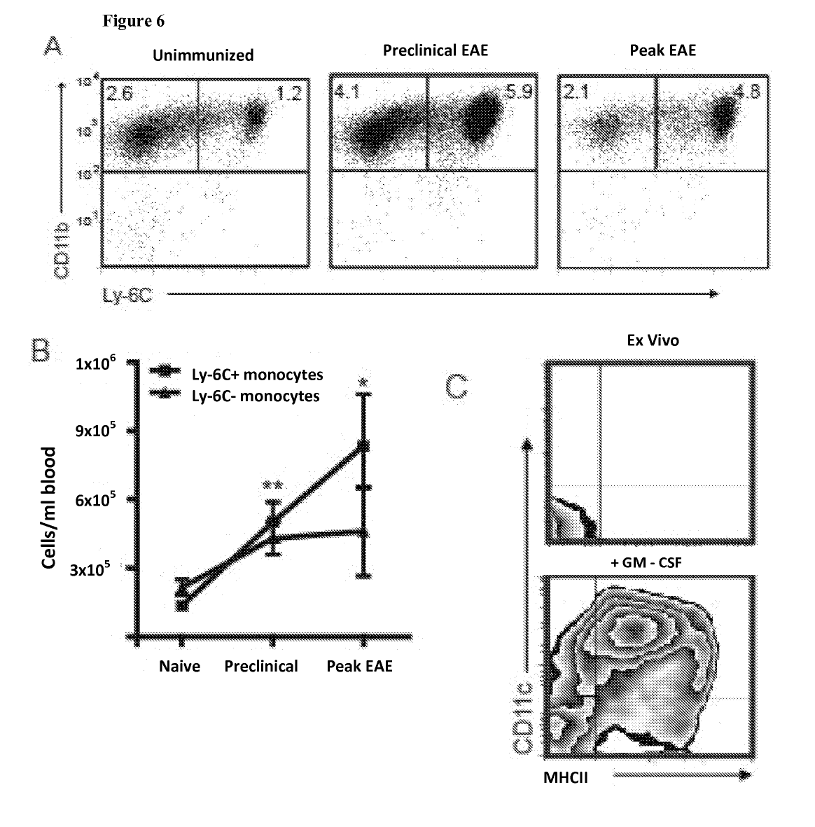

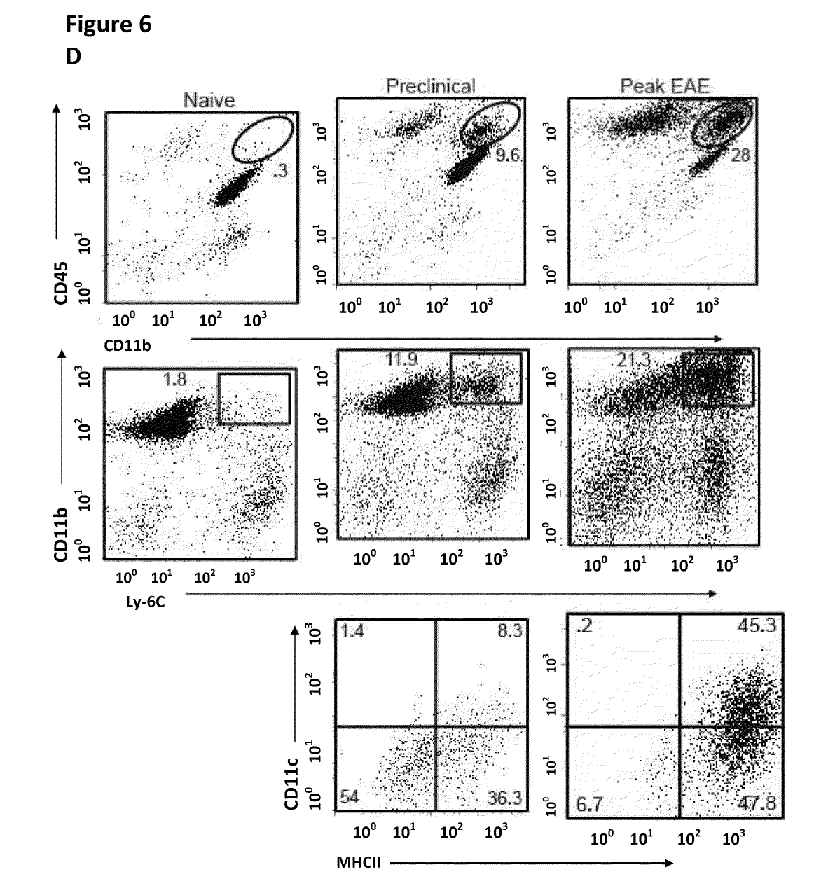

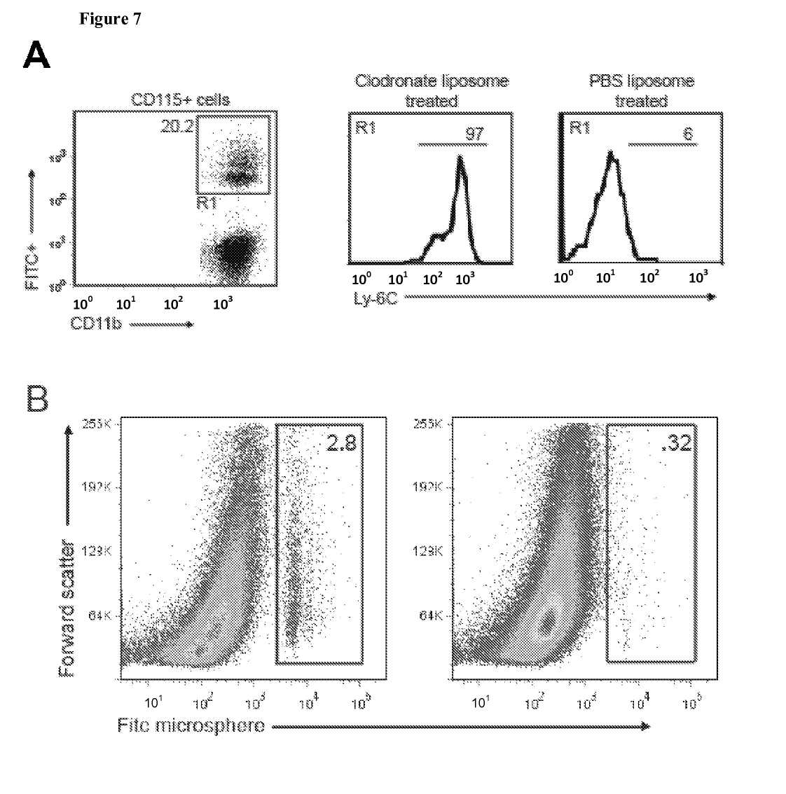

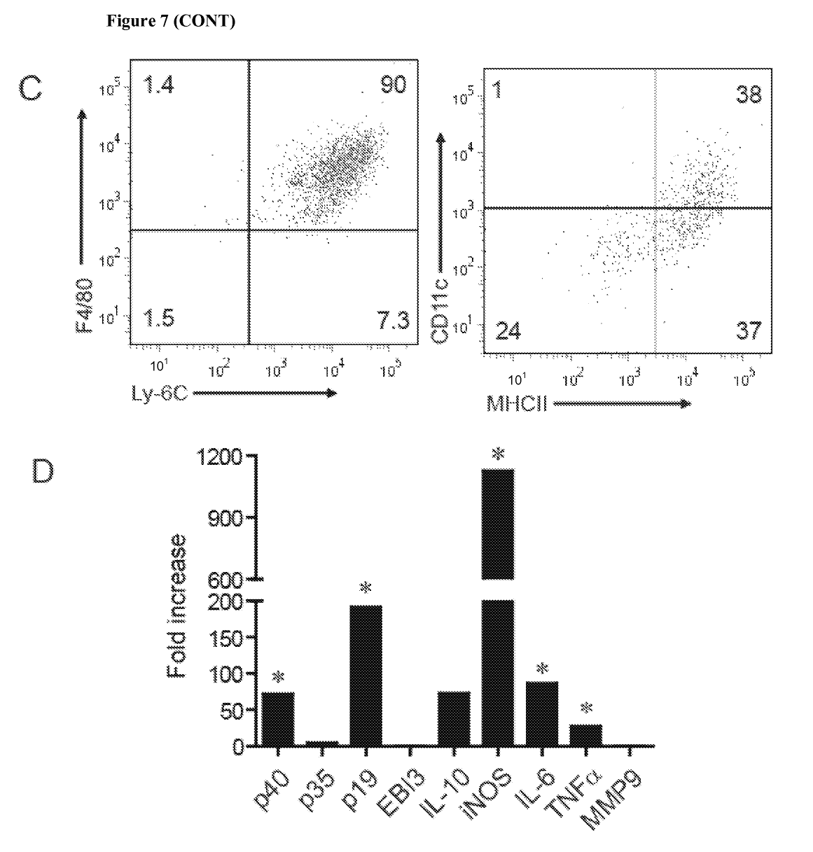

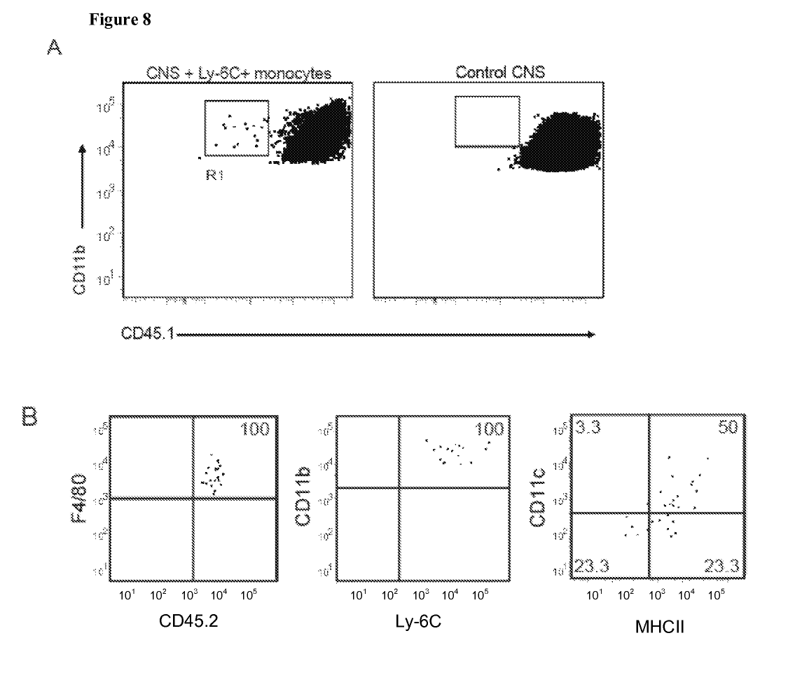

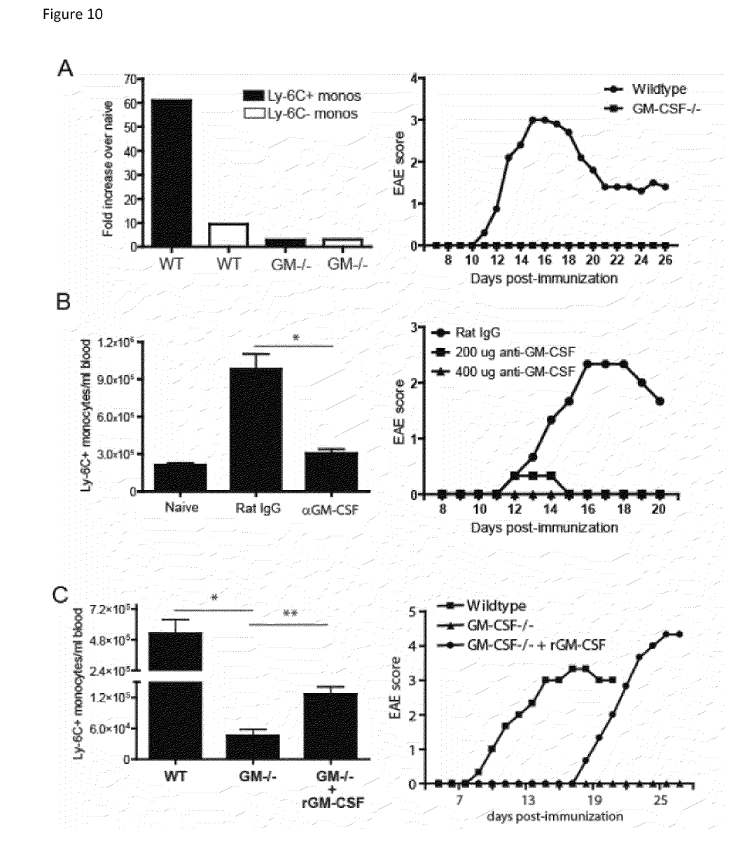

The frequency of granulocyte/monocyte colony-forming units (GM-CFU) rises in the circulation of myelin-immunized mice immediately prior to clinical episodes of EAE (FIG. 5). GM-CFU represent myeloid precursor cells. GM-CFU activity is contained within the CD11b⁺CD62L⁺Ly-6C^{hi} subset of peripheral monocytes (FIG. 6). Furthermore, circulating CD11b⁺Ly-6C^{hi} white blood cells migrate to the CNS during EAE and upregulate CD11c and MHC Class II in situ (FIGS. 7 and 8). The CD11b⁺Ly-6C^{hi} subset corresponds to recently described CX3CR1loGr−1+Ly6ChiCD62L+ “inflammatory” monocytes (iM) that preferentially home to inflamed tissue (Geissmann et al., Immunity 19, 71-82 (2003)). EAE is more severe and occurs after a shorter latency under conditions favoring the enrichment of circulating CD11b⁺Ly-6C^{hi} cells (FIG. 10). Furthermore, administration of recombinant GM-CSF to GM-CSF deficient animals triggers CD11b⁺Ly-6C^{hi} mobilization and confers susceptibility to EAE (FIG. 11).

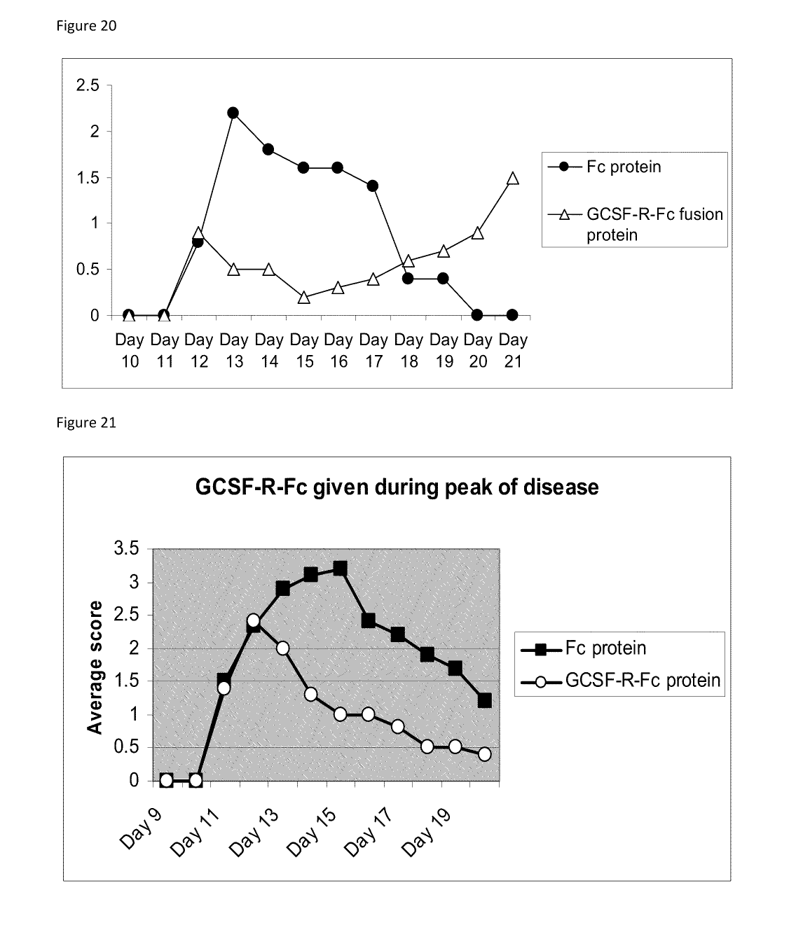

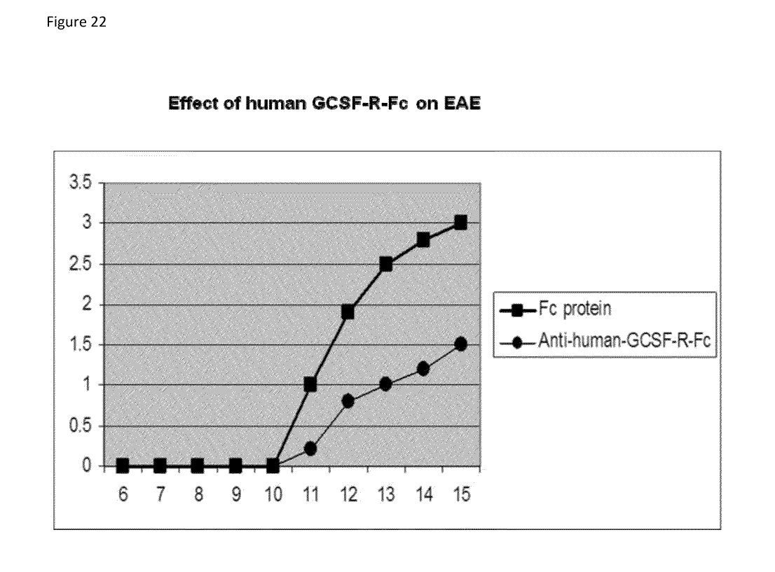

Mobilization of myeloid cell precursors into the circulation can be driven by G-CSF mediated activation of neutrophils in the bone marrow (Pelus, et al. Exp Hematol. 34, 1010-1020 (2006)). In this capacity G-CSF often acts in synergy with other growth factors (GM-CSF) and chemokines (CXCL1, CXCL2) (Lonial et al., Biol Blood Marrow Transplant 10, 848-857 (2004)). G-CSF directly stimulates bone marrow neutrophils to secrete proteases that degrade chemokines (such as CXCL12) and adhesion molecules (such as α4β1 integrin and VCAM-1) that normally keep myeloid cells “anchored” within intramedullary niches (Christopher et al., Blood (2009); Lapidot et al., Exp Hematol 30, 973-981 (2002); Levesque et al., J Clin Invest 111, 187-196 (2003); Levesque et al., Blood 98, 1289-1297 (2001); Petit et al., Nat Immunol 3, 687-694 (2002)). Experiments conducted during the course of development of embodiments of the present invention indicated that G-CSF blockade results in the amelioration of clinical EAE (FIGS. 20-22). Administration of recombinant granulocyte colony stimulating factor (G-CSF) to patients with MS undergoing experimental bone marrow transplantation was associated with the development of severe clinical exacerbations (Openshaw et al., Neurology 54, 2147-2150 (2000)). The present invention is not limited to a particular mechanism. Indeed, an understanding of the mechanism is not necessary to practice the present invention. Nonetheless, it is contemplated that G-CSF contributes to lesion formation and tissue damage during neuroinflammatory disease, in part, by enhancing the release of CNS macrophage and dendritic cells precursors into the bloodstream immediately prior to clinical exacerbations.

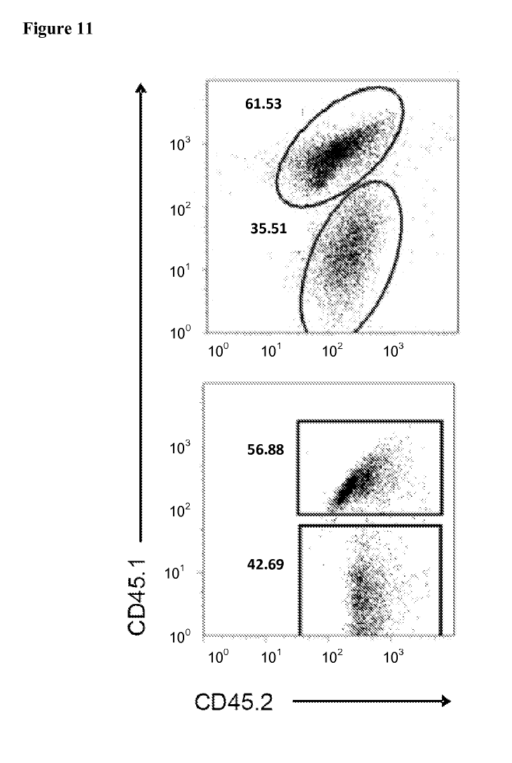

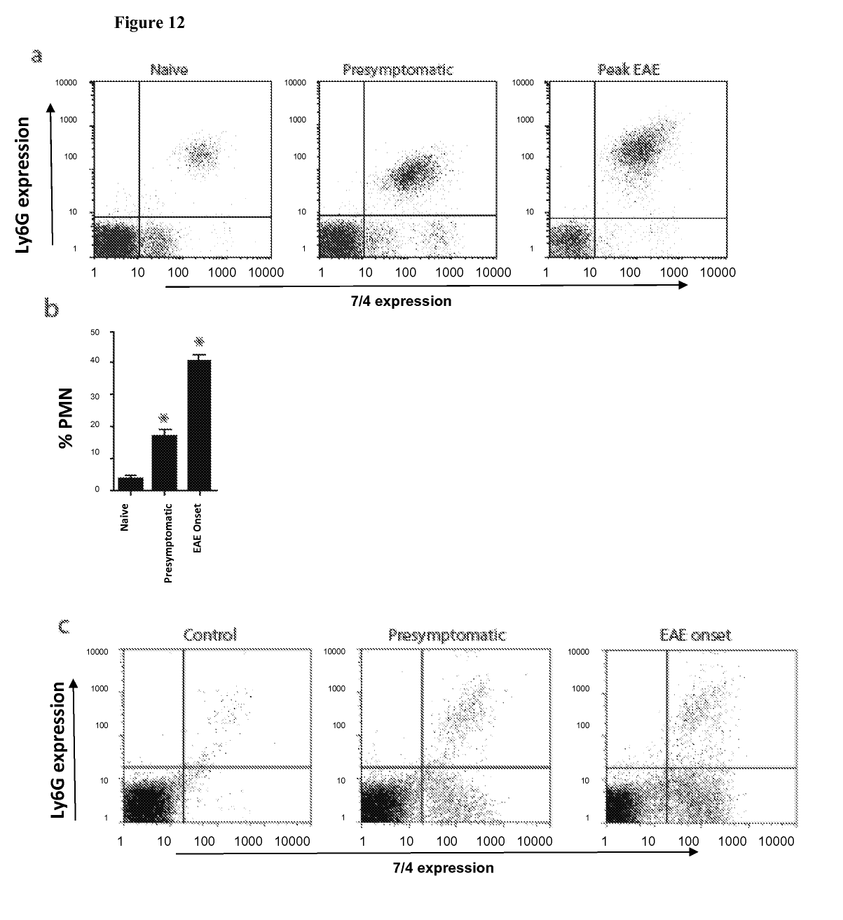

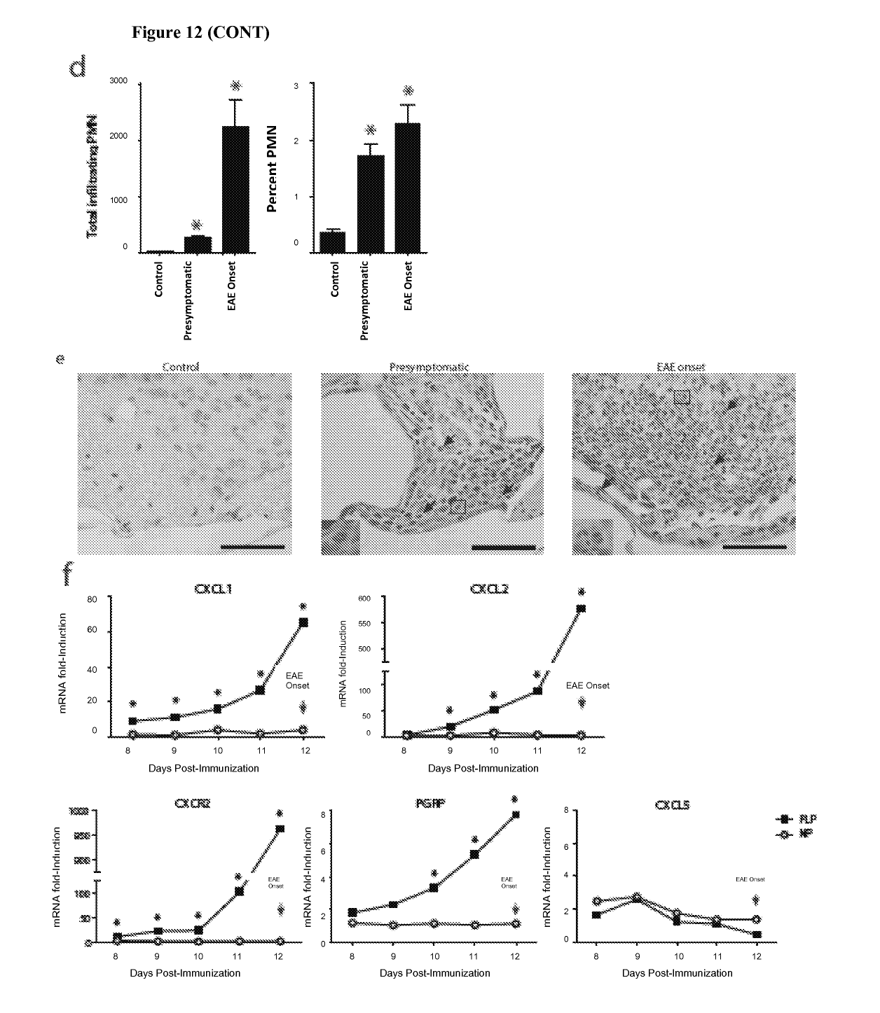

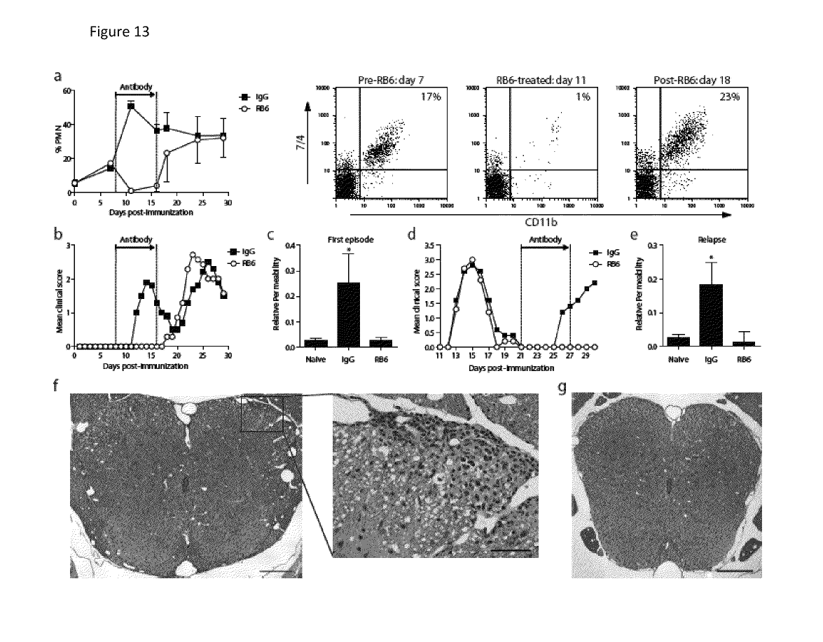

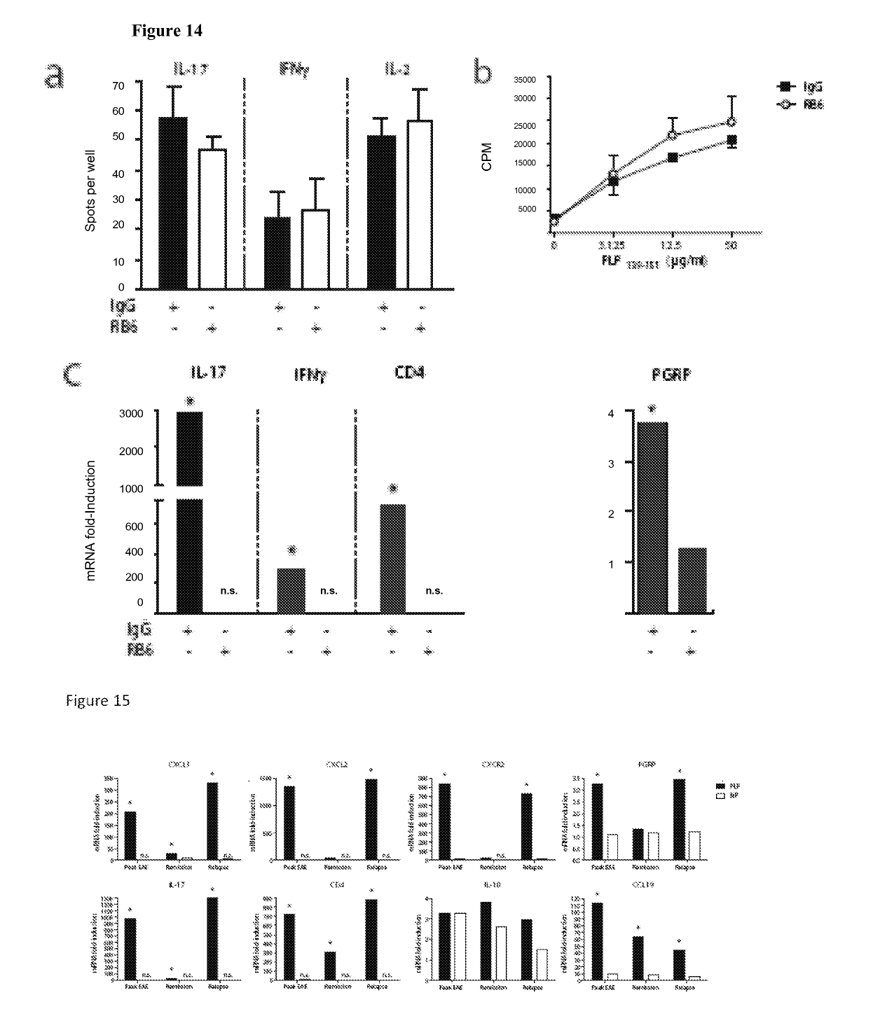

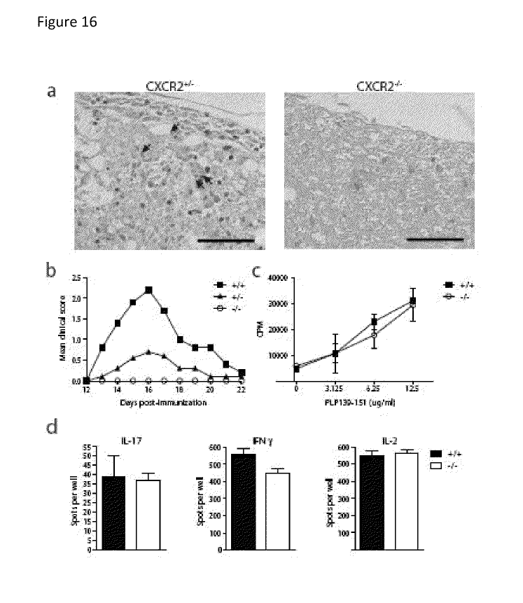

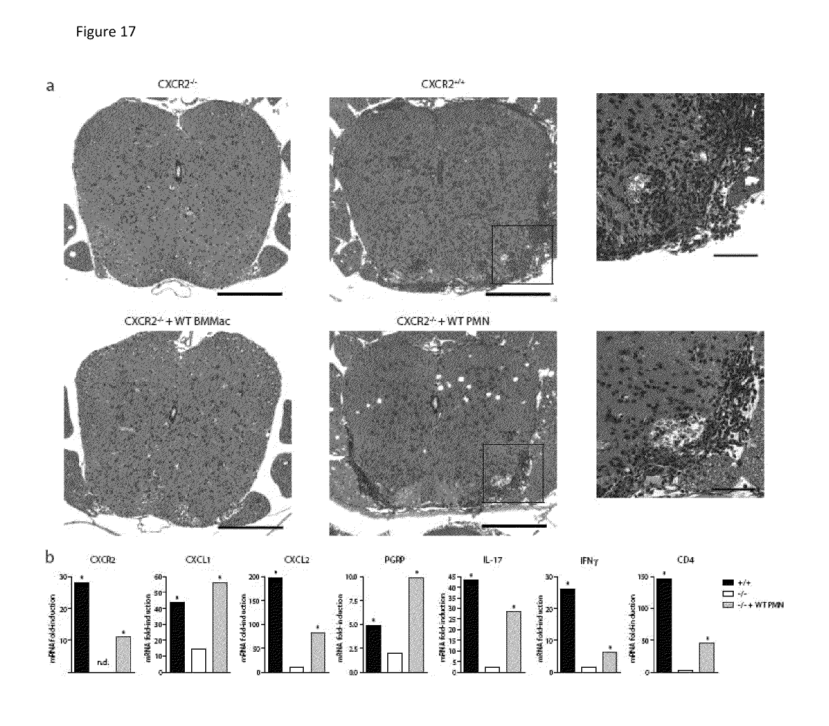

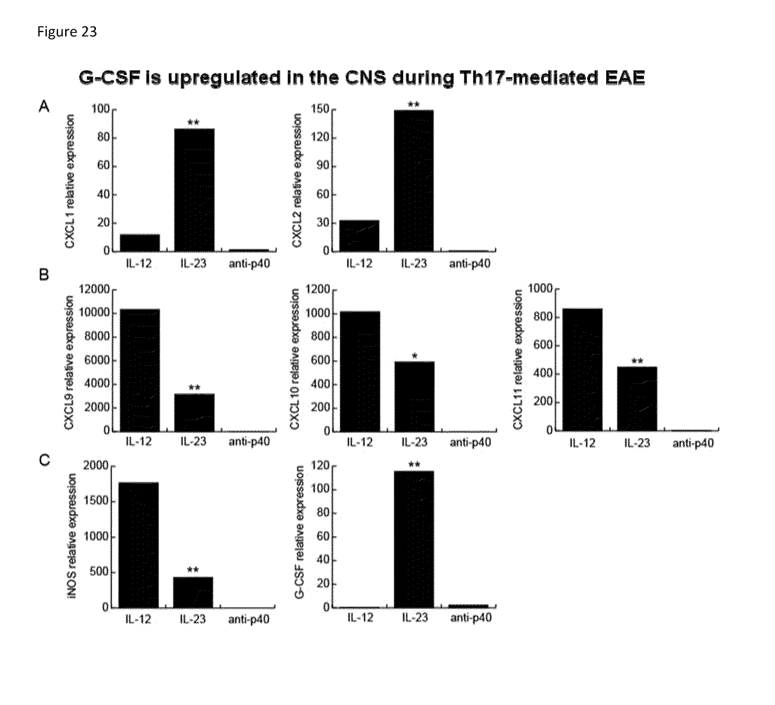

The present invention is not limited to a particular mechanism. Indeed, an understanding of the mechanism is not necessary to practice the present invention. Nonetheless, it is contemplated that G-CSF also promotes disease activity via activation of neutrophils within the target organ, and/or within blood vessels supplying the target organ, to release factors that increase vascular permeability and degrade the extracellular matrix, thereby enhancing infiltration of leukocytes into the nascent lesion (Del Maschio, et al. J. Cell. Biol. 135, 497-510 (1996)). Blood-brain barrier (BBB) breakdown is a critical step bridging the reactivation of myelin-specific T cells within the CNS to the massive influx of non-specific leukocytes that heralds the onset of clinical disability. Blood-brain-barrier breakdown occurs in association with PMN infiltration of the CNS provoked by either viral encephalitis, intracerebral injection of IL-1β or CXCL2, or by transgenic expression of CXCL1 in oligodendrocytes (Zhou, et. al. J. Immunol. 170, 3331-3336 (2003); Anthony, et al. Brain 120 (Pt 3), 435-444 (1997); Bell, et. al. Neuroscience 74, 283-292 (1996); Tani, et al. J. Clin. Invest. 98, 529-539 (1996)). Neutrophils are among the first leukocytes to enter the CNS during EAE and are present in established infiltrates in small numbers (Brown, et. al. Lab. Invest. 46, 171-185 (1982); FIG. 12). Furthermore, G-CSF and other neutrophil associated factors are upregulated in EAE and MS lesions (Kroenke, et al. J Exp Med 205, 1535-1541 (2008); Lock, et al. Nat. Med. 8, 500-508 (2002); FIGS. 15 and 23). Experiments conducted during the course of development of embodiments of the present invention demonstrated that depletion of neutrophils prevents blood-brain-barrier disruption, the upregulation of inflammatory markers in the CNS and the clinical and histological manifestations of EAE in myelin-immunized mice (FIGS. 13 and 14). Mice deficient in CXCR2, a chemoattractant receptor expressed on neutrophils, are resistant to EAE but are rendered susceptible by reconstitution with wildtype neutrophils (FIGS. 16 and 17). Collectively, these data indicate that activation of neutrophils is a crucial step in the development of neuroinflammation. G-CSF, a potent neutrophil activating factor that is upregulated in the CNS during EAE and MS (FIG. 23), plays a role in this process.

### I. G-CSFR Targeted Therapeutics

In some embodiments, the present invention provides compositions and methods for altering G-CSF-G-CSF-R interactions. The present invention is not limited to a particular therapy. In some embodiments, G-CSF and/or G-CSF-R activity, binding characteristics, or expression are altered by an agent to generate the desired result. Exemplary therapeutic compositions and methods are described below. The invention is not limited to these particular agents or their mechanisms of action.

A. FC-Fusion Therapy

In some embodiments, FC-fusion molecules are utilized. These embodiments are exemplified by G-CSFR-FC-fusions. However, the invention is not limited to these embodiments. Fc-fusion proteins are chimeric proteins comprising the effector region of a protein (e.g., G-CSFR), fused to the Fc region of an immunoglobulin G (IgG).

The G-CSFR fragment of the Fc fusion molecule can be any suitable G-CSFR fragment. In some embodiments, the fragment is an extracellular domain of G-CSFR. Alternatively, a variant of a G-CSFR fragment described above can be used. Desirably, the variant of the G-CSFR fragment retains the functionality of the selected fragment. A variant of a G-CSFR fragment can be obtained by any suitable method, including random and site-directed mutagenesis of the nucleic acid encoding the G-CSFR fragment (see, e.g., Walder et al., Gene, 42, 133 (1986); Bauer et al., Gene, 37, 73 (1985); U.S. Pat. Nos. 4,518,584 and 4,732,462; and QuikChange Site-Directed Mutagenesis Kit (Stratagene, LaJolla, Calif.); each of which is herein incorporated by reference in its entirety). While a variant of the nucleic acid can be generated in vivo and then isolated and purified, alternatively, a variant of the nucleic acid can be synthesized. Various techniques used to synthesize nucleic acids are known in the art (see, e.g., Lemaitre et al., Proc. Natl. Acad. Sci., 84, 648-652 (1987)).

Additionally, a variant can be synthesized using peptide-synthesizing techniques known in the art (see, e.g., Bodansky, Principles of Peptide Synthesis, Springer-Verlag, Heidelberg, 1984). In particular, a (poly)peptide can be synthesized using the procedure of solid-phase synthesis (see, e.g., Merrifield, J. Am. Chem. Soc., 85, 2149-54 (1963); Barany et al., Int. J. Peptide Protein Res., 30, 705-739 (1987), and U.S. Pat. No. 5,424,398; each of which is herein incorporated by reference in its entirety). If desired, a (poly)peptide can be synthesized with an automated peptide synthesizer. Removal of the t-butyloxycarbonyl (t-BOC) or 9-fluorenylmethyloxycarbonyl (Fmoc) amino acid blocking groups and separation of the (poly)peptide from the resin can be accomplished by, for example, acid treatment at reduced temperature. The (poly)peptide-containing mixture can then be extracted, for instance, with dimethyl ether, to remove non-peptidic organic compounds, and the synthesized (poly)peptide can be extracted from the resin powder (e.g., with about 25% w/v acetic acid). Following the synthesis of the (poly)peptide, further purification (e.g., using high performance liquid chromatography (HPLC)) optionally can be done in order to eliminate any incomplete (poly)peptides or free amino acids. Amino acid and/or HPLC analysis can be performed on the synthesized polypeptide to determine its identity. The (poly)peptide can be produced as part of a larger fusion protein, such as by the above-described methods or genetic means, or as part of a larger conjugate, such as through physical or chemical conjugation.

The variant of the above-described G-CSFR fragment includes molecules that have about 50% or more identity to the above-described G-CSFR fragments. Preferably, the variant includes molecules that have 75% identity to the above-described G-CSFR fragments. More preferably, the variant includes molecules that have 85% (e.g., about 90% or more, about 95% or more, about 96% or more, about 97% or more, about 98% or more, or about 99% or more) identity with the above-described G-CSFR fragments. Ideally, the variant of the G-CSFR fragment contains from 1 to about 40 (e.g., about 5, about 10, about 15, about 20, about 25, about 30, about 35, or ranges thereof) amino acid substitutions, deletions, inversions, and/or insertions thereof. More preferably, the variant of the above-described G-CSFR fragments contains from 1 to about 20 amino acid substitutions, deletions, inversions, and/or insertions thereof. Most preferably, the variant of the scFv fragment contains from 1 to about 10 amino acid substitutions, deletions, inversions, and/or insertions thereof.

The substitutions, deletions, inversion, and/or insertions of the G-CSFR fragment preferably occur in non-essential regions. The identification of essential and non-essential amino acids in the G-CSFR fragment can be achieved by methods known in the art, such as by site-directed mutagenesis and AlaScan analysis (see, e.g., Moffison et al., Chem. Biol. 5(3), 302-307 (2001)). Essential amino acids should be maintained or replaced by conservative substitutions in the variants of the G-CSFR fragments. Non-essential amino acids can be deleted, or replaced by a spacer or by conservative or non-conservative substitutions.

The variants can be obtained by substitution of any of the amino acids as present in the G-CSFR fragment. As can be appreciated, there are positions in the sequence that are more tolerant to substitutions than others, and some substitutions can improve the activity of the native G-CSFR fragment. The amino acids that are essential should either be identical to the amino acids present in the G-CSFR fragment, or substituted by conservative substitutions. The amino acids that are nonessential can be identical to those in the G-CSFR fragment, can be substituted by conservative or non-conservative substitutions, and/or can be deleted.

Conservative substitution refers to the replacement of an amino acid in the G-CSFR fragment with a naturally or non-naturally occurring amino acid having similar steric properties. Where the side-chain of the amino acid to be replaced is either polar or hydrophobic, the conservative substitution should be with a naturally or non-naturally occurring amino acid that is also polar or hydrophobic (in addition to having the same steric properties as the side-chain of the replaced amino acid). When the native amino acid to be replaced is charged, the conservative substitution can be with a naturally or non-naturally occurring amino acid that is charged, or with a non-charged (polar, hydrophobic) amino acid that has the same steric properties as the side-chains of the replaced amino acid. For example, the replacement of arginine by glutamine, aspartate by asparagine, or glutamate by glutamine is considered to be a conservative substitution.

In order to further exemplify what is meant by conservative substitution, Groups A-F are listed below. The replacement of one member of the following groups by another member of the same group is considered to be a conservative substitution.

Group A includes leucine, isoleucine, valine, methionine, phenylalanine, serine, cysteine, threonine, and modified amino acids having the following side chains: ethyl, iso-butyl, —CH2CH2OH, —CH2CH2CH2OH, —CH2CHOHCH3 and CH2SCH3.

Group B includes glycine, alanine, valine, serine, cysteine, threonine, and a modified amino acid having an ethyl side chain.

Group C includes phenylalanine, phenylglycine, tyrosine, tryptophan, cyclohexylmethyl, and modified amino residues having substituted benzyl or phenyl side chains.

Group D includes glutamic acid, aspartic acid, a substituted or unsubstituted aliphatic, aromatic or benzylic ester of glutamic or aspartic acid (e.g., methyl, ethyl, n-propyl, iso-propyl, cyclohexyl, benzyl, or substituted benzyl), glutamine, asparagine, CO—NH-alkylated glutamine or asparagine (e.g., methyl, ethyl, n-propyl, and iso-propyl), and modified amino acids having the side chain —(CH2)3COOH, an ester thereof (substituted or unsubstituted aliphatic, aromatic, or benzylic ester), an amide thereof, and a substituted or unsubstituted N-alkylated amide thereof.

Group E includes histidine, lysine, arginine, N-nitroarginine, p-cycloarginine, g-hydroxyarginine, N-amidinocitruiine, 2-amino guanidinobutanoic acid, homologs of lysine, homologs of arginine, and ornithine.

Group F includes serine, threonine, cysteine, and modified amino acids having C1-C5 straight or branched alkyl side chains substituted with —OH or —SH.

A non-conservative substitution is a substitution in which the substituting amino acid (naturally or non-naturally occurring) has significantly different size, configuration and/or electronic properties compared with the amino acid being substituted. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cyclohexylmethyl glycine for alanine, or isoleucine for glycine. Alternatively, a functional group can be added to the side chain, deleted from the side chain or exchanged with another functional group. Examples of nonconservative substitutions of this type include adding an amine, hydroxyl, or carboxylic acid to the aliphatic side chain of valine, leucine or isoleucine, or exchanging the carboxylic acid in the side chain of aspartic acid or glutamic acid with an amine or deleting the amine group in the side chain of lysine or ornithine.

For non-conservative substitutions, the side chain of the substituting amino acid can have significantly different steric and electronic properties from the functional group of the amino acid being substituted. Examples of such modifications include tryptophan for glycine, and lysine for aspartic acid.

The Fc region of the fusion molecule can be any suitable Fc region of an antibody. Preferably, the Fc region increases the stability, decreases the clearance time of the peptide or polypeptide (e.g., G-CSFR fragment) from plasma and tissues, thereby enabling a minimum effective dose to be realized. The Fc region of an antibody is limited in variability and is responsible for the biological effector function of the antibody, which is designed to block the activity of G-CSFR. The Fc portion varies between antibody classes (and subclasses) but is identical within that class. If the Fc region is a human Fc region, the Fc region is selected from the classes of IgA, IgD, IgE, IgG, and IgM. If the Fc region is an IgA or IgG Fc region, the subclass is selected from IgA1 and IgA2, or IgG1, IgG2, IgG3, and IgG4, respectively. In some embodiments, the Fc region is an Fc region of IgG. In some embodiments, the Fc region is an IgG2A heavy chain (CH2 and CH3 domains and the hinge region).

The peptide or polypeptide (e.g., G-CSF fragment) and Fc region of the fusion molecule optionally are joined together by a linker. The linker can be any suitable long flexible linker. The linker can be any suitable length, but is preferably at least about 15 (e.g., at least about 20, at least about 25, at least about 30, at least about 35, at least about 40, at least about 45, at least about 50, or ranges thereof) amino acids in length. Preferably, the long flexible linker is an amino acid sequence that is naturally present in immunoglobulin molecules of the host, such that the presence of the linker would not result in an immune response against the linker sequence by the mammal.

The generation of the fusion molecules of the invention is within the ordinary skill in the art, and can comprise the use of restriction enzyme or recombinational cloning techniques (see, e.g., Gateway™ (Invitrogen), Invivogen and U.S. Pat. No. 5,314,995; herein incorporated by reference in its entirety).

As discussed above, the Fc fusion molecules encompassed by the invention comprise a G-CSFR fragment, an Fc region, and, optionally, a flexible linker. Further, any of these fusion molecules can be expressed with a suitable leader sequence, which leader sequence specifies how the fusion is trafficked through a cell expressing the leader-fusion polypeptide. In some embodiments, fusion protein includes a signal sequence such as an IL-2 signal sequence (IL-2ss).

Variants of the Fc fusion molecules can be obtained by any suitable method, including those methods discussed above. The variants of the above-described Fc fusion molecules include molecules that have about 90% or more percent identity (e.g., about 95% or more, about 96% or more, about 97% or more, about 98% or more, or about 99% or more) with the above-described Fc fusion molecules. Preferably, the variants of the Fc fusion molecules contain from 1 to about 50 (e.g., about 5, about 10, about 15, about 20, about 25, about 30, about 35, about 40, about 45, or ranges thereof) amino acid substitutions, deletions, inversions, and/or insertions thereof. More preferably, the variants contain from 1 to about 30 amino acid substitutions, deletions, inversions, and/or insertions thereof. Most preferably, the variants contain from 1 to about 20 amino acid substitutions, deletions, inversions, and/or insertions thereof. Ideally, the variants contain from 1 to about 10 amino acid substitutions, deletions, inversions, and/or insertions thereof. Preferably, the Fc region of the Fc fusion molecule and nucleotides encoding the same remain unchanged or are only slightly changed, such as by conservative or neutral amino acid substitution(s).

The Fc fusion molecule preferably disrupts the interaction between G-CSF and G-CSFR, and thus reduces, prevents, or eliminates symptoms of a disease state (e.g., MS or related neuroinflammatory conditions).

B. Antibody Therapy

In some embodiments, the present invention provides antibodies that alter G-CSF-G-CSFR interactions (e.g., by binding to and neutralizing G-CSF or by binding to and blocking G-CSFR) and thus reduce, prevent or eliminate symptoms of a disease state (e.g., MS or related neuroinflammatory conditions). Any suitable antibody (e.g., monoclonal, polyclonal, or synthetic) may be utilized in the therapeutic methods disclosed herein. In preferred embodiments, the antibodies used for therapy are humanized antibodies. Methods for humanizing antibodies are well known in the art (See e.g., U.S. Pat. Nos. 6,180,370, 5,585,089, 6,054,297, and 5,565,332; each of which is herein incorporated by reference).

In some embodiments, the therapeutic antibodies comprise an antibody generated against G-CSFR, wherein the antibody is conjugated to a cytotoxic agent. For certain applications, it is envisioned that the therapeutic agents will be pharmacologic agents that will serve as useful agents for attachment to antibodies, particularly cytotoxic or otherwise anticellular agents having the ability to kill or suppress the growth or cell division of target cells. The present invention contemplates the use of any pharmacologic agent that can be conjugated to an antibody, and delivered in active form. Exemplary anticellular agents include chemotherapeutic agents, radioisotopes, and cytotoxins. The therapeutic antibodies of the present invention may include a variety of cytotoxic moieties, including but not limited to, radioactive isotopes (e.g., iodine-131, iodine-123, technicium-99m, indium-111, rhenium-188, rhenium-186, gallium-67, copper-67, yttrium-90, iodine-125 or astatine-211), hormones such as a steroid, antimetabolites such as cytosines (e.g., arabinoside, fluorouracil, methotrexate or aminopterin; an anthracycline; mitomycin C), vinca alkaloids (e.g., demecolcine; etoposide; mithramycin), and antitumor alkylating agent such as chlorambucil or melphalan. Other embodiments may include agents such as a cytokine, growth factor, bacterial endotoxin or the lipid A moiety of bacterial endotoxin. For example, in some embodiments, therapeutic agents will include plant-, fungus- or bacteria-derived toxin, such as an A chain toxins, a ribosome inactivating protein, α-sarcin, aspergillin, restrictocin, a ribonuclease, diphtheria toxin or pseudomonas exotoxin, to mention just a few examples. In some preferred embodiments, deglycosylated ricin A chain is utilized.

In any event, it is proposed that agents such as these may, if desired, be successfully conjugated to an antibody, in a manner that will allow their targeting, internalization, release or presentation granulocytes as required using known conjugation technology (See, e.g., Ghose et al., Methods Enzymol., 93:280 [1983]).

For example, in some embodiments the present invention provides immunotoxins targeting G-CSFR. Immunotoxins are conjugates of a specific targeting agent typically an antibody or fragment, with a cytotoxic agent, such as a toxin moiety. The targeting agent directs the toxin to, and thereby selectively kills, cells carrying the targeted antigen. In some embodiments, therapeutic antibodies employ crosslinkers that provide high in vivo stability (Thorpe et al., Cancer Res., 48:6396 [1988]).

In preferred embodiments, antibody based therapeutics are formulated as pharmaceutical compositions as described below. In preferred embodiments, administration of an antibody composition of the present invention results in a measurable decrease in symptoms of a disease state (e.g., MS or other neuroinflammatory disease).

C. Nucleic Acid Therapeutics

In some embodiments, nucleic acid based therapeutics are utilized. In some embodiments, therapeutics are nucleic acid based (e.g., siRNA or antisense).

1. RNA Interference (RNAi)

In some embodiments, RNAi is utilized to inhibit G-CSF, G-CSFR or G-CSFR signaling function. RNAi represents an evolutionary conserved cellular defense for controlling the expression of foreign genes in most eukaryotes, including humans. RNAi is typically triggered by double-stranded RNA (dsRNA) and causes sequence-specific mRNA degradation of single-stranded target RNAs homologous in response to dsRNA. The mediators of mRNA degradation are small interfering RNA duplexes (siRNAs), which are normally produced from long dsRNA by enzymatic cleavage in the cell. siRNAs are generally approximately twenty-one nucleotides in length (e.g. 21-23 nucleotides in length), and have a base-paired structure characterized by two nucleotide 3′-overhangs. Following the introduction of a small RNA, or RNAi, into the cell, it is believed the sequence is delivered to an enzyme complex called RISC (RNA-induced silencing complex). RISC recognizes the target and cleaves it with an endonuclease. It is noted that if larger RNA sequences are delivered to a cell, RNase III enzyme (Dicer) converts longer dsRNA into 21-23 nt ds siRNA fragments.

Chemically synthesized siRNAs have become powerful reagents for genome-wide analysis of mammalian gene function in cultured somatic cells. Beyond their value for validation of gene function, siRNAs also hold great potential as gene-specific therapeutic agents (Tuschl and Borkhardt, Molecular Intervent. 2002; 2(3):158-67, herein incorporated by reference).

The transfection of siRNAs into animal cells results in the potent, long-lasting post-transcriptional silencing of specific genes (Caplen et al, Proc Natl Acad Sci U.S.A. 2001; 98: 9742-7; Elbashir et al., Nature. 2001; 411:494-8; Elbashir et al., Genes Dev. 2001; 15: 188-200; and Elbashir et al., EMBO J. 2001; 20: 6877-88, all of which are herein incorporated by reference). Methods and compositions for performing RNAi with siRNAs are described, for example, in U.S. Pat. No. 6,506,559, herein incorporated by reference.

siRNAs are extraordinarily effective at lowering the amounts of targeted RNA, and by extension proteins, frequently to undetectable levels. The silencing effect can last several months, and is extraordinarily specific, because one nucleotide mismatch between the target RNA and the central region of the siRNA is frequently sufficient to prevent silencing (Brummelkamp et al, Science 2002; 296:550-3; and Holen et al, Nucleic Acids Res. 2002; 30:1757-66, both of which are herein incorporated by reference).

An important factor in the design of siRNAs is the presence of accessible sites for siRNA binding. Bahoia et al., (J. Biol. Chem., 2003; 278: 15991-15997; herein incorporated by reference) describe the use of a type of DNA array called a scanning array to find accessible sites in mRNAs for designing effective siRNAs. These arrays comprise oligonucleotides ranging in size from monomers to a certain maximum, usually Comers, synthesized using a physical barrier (mask) by stepwise addition of each base in the sequence. Thus the arrays represent a full oligonucleotide complement of a region of the target gene. Hybridization of the target mRNA to these arrays provides an exhaustive accessibility profile of this region of the target mRNA. Such data are useful in the design of antisense oligonucleotides (ranging from 7mers to 25mers), where it is important to achieve a compromise between oligonucleotide length and binding affinity, to retain efficacy and target specificity (Sohail et al, Nucleic Acids Res., 2001; 29(10): 2041-2045). Additional methods and concerns for selecting siRNAs are described for example, in WO 05054270, WO05038054A1, WO03070966A2, J Mol. Biol. 2005 May 13; 348(4):883-93, J Mol. Biol. 2005 May 13; 348(4):871-81, and Nucleic Acids Res. 2003 Aug. 1; 31(15):4417-24, each of which is herein incorporated by reference in its entirety. In addition, software (e.g., the MWG online siMAX siRNA design tool) is commercially or publicly available for use in the selection of siRNAs.

2. Antisense

In other embodiments, G-CSF, G-CSFR or G-CSFR signaling partner expression is modulated using antisense compounds that specifically hybridize with one or more nucleic acids encoding G-CSF or G-CSFR of the present invention. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of G-CSF or G-CSFR of the present invention. In the context of the present invention, “modulation” means either an increase (stimulation) or a decrease (inhibition) in the expression of a gene. For example, expression may be inhibited to potentially prevent tumor proliferation.

It is preferred to target specific nucleic acids for antisense. “Targeting” an antisense compound to a particular nucleic acid, in the context of the present invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding a G-CSF or G-CSFR of the present invention. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). Eukaryotic and prokaryotic genes may have two or more alternative start codons, any one of which may be preferentially utilized for translation initiation in a particular cell type or tissue, or under a particular set of conditions. In the context of the present invention, “start codon” and “translation initiation codon” refer to the codon or codons that are used in vivo to initiate translation of an mRNA molecule transcribed from a gene encoding a tumor antigen of the present invention, regardless of the sequence(s) of such codons.

Translation termination codon (or “stop codon”) of a gene may have one of three sequences (i.e., 5′-UAA, 5′-UAG and 5′-UGA; the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which refers to the region between the translation initiation codon and the translation termination codon, is also a region that may be targeted effectively. Other target regions include the 5′ untranslated region (5′ UTR), referring to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′ UTR), referring to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The cap region may also be a preferred target region.

Although some eukaryotic mRNA transcripts are directly translated, many contain one or more regions, known as “introns,” that are excised from a transcript before it is translated. The remaining (and therefore translated) regions are known as “exons” and are spliced together to form a continuous mRNA sequence. mRNA splice sites (i.e., intron-exon junctions) may also be preferred target regions, and are particularly useful in situations where aberrant splicing is implicated in disease, or where an overproduction of a particular mRNA splice product is implicated in disease. It has also been found that introns can also be effective, and therefore preferred, target regions for antisense compounds targeted, for example, to DNA or pre-mRNA.

In some embodiments, target sites for antisense inhibition are identified using commercially available software programs (e.g., Biognostik, Gottingen, Germany; SysArris Software, Bangalore, India; Antisense Research Group, University of Liverpool, Liverpool, England; GeneTrove, Carlsbad, Calif.). In other embodiments, target sites for antisense inhibition are identified using the accessible site method described in PCT Publ. No. WO0198537A2, herein incorporated by reference.

Once one or more target sites have been identified, oligonucleotides are chosen that are sufficiently complementary to the target (i.e., hybridize sufficiently well and with sufficient specificity) to give the desired effect. For example, in preferred embodiments of the present invention, antisense oligonucleotides are targeted to or near the start codon.

In the context of this invention, “hybridization,” with respect to antisense compositions and methods, means hydrogen bonding, which may be Watson-Crick, Hoogsteen or reversed Hoogsteen hydrogen bonding, between complementary nucleoside or nucleotide bases. For example, adenine and thymine are complementary nucleobases that pair through the formation of hydrogen bonds. It is understood that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired (i.e., under physiological conditions in the case of in vivo assays or therapeutic treatment, and in the case of in vitro assays, under conditions in which the assays are performed).

The specificity and sensitivity of antisense is also applied for therapeutic uses. For example, antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides are useful therapeutic modalities that can be configured to be useful in treatment regimes for treatment of cells, tissues, and animals, especially humans.

While antisense oligonucleotides are a preferred form of antisense compound, the present invention comprehends other oligomeric antisense compounds, including but not limited to oligonucleotide mimetics. The antisense compounds in accordance with this invention preferably comprise from about 8 to about 30 nucleobases (i.e., from about 8 to about 30 linked bases), although both longer and shorter sequences may find use with the present invention. Particularly preferred antisense compounds are antisense oligonucleotides, even more preferably those comprising from about 12 to about 25 nucleobases.

D. Small Molecule Therapies

In other embodiments, the present invention provides small molecule inhibitors of G-CSF or G-CSFR expression, activity, or interactions between G-CSF and G-CSFR. In some embodiments, small molecule therapeutics are identified using drug screening methods (e.g., those described herein).

E. Gene Therapy

The present invention contemplates the use of any genetic manipulation for use in modulating the expression of G-CSF, G-CSFR or G-CSFR signaling partners. Examples of genetic manipulation include, but are not limited to, gene knockout (e.g., removing the G-CSF, G-CSFR or G-CSFR signaling molecule from the chromosome using, for example, recombination), expression of antisense constructs with or without inducible promoters, and the like. Delivery of nucleic acid construct to cells in vitro or in vivo may be conducted using any suitable method. A suitable method is one that introduces the nucleic acid construct into the cell such that the desired event occurs (e.g., expression of an antisense construct). Genetic therapy may also be used to deliver siRNA or other interfering molecules that are expressed in vivo (e.g., upon stimulation by an inducible promoter).

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Preferred methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer. Examples of adenoviral vectors and methods for gene transfer are described in PCT publications WO 00/12738 and WO 00/09675 and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

Vectors may be administered to subject in a variety of ways. For example, in some embodiments of the present invention, vectors are administered into tumors or tissue associated with tumors using direct injection. In other embodiments, administration is via the blood or lymphatic circulation (See e.g., PCT publication 99/02685 herein incorporated by reference in its entirety). Exemplary dose levels of adenoviral vector are preferably 10⁸ to 10¹¹ vector particles added to the perfusate.

### II. Therapeutic Applications

As described above, embodiments of the present invention provide compositions and method for treating neuroinflammatory diseases (e.g., MS) associated with aberrant G-CSF or G-CSFR expression and/or signaling.

A. Therapeutic Methods

Embodiments of the present invention provide methods of treating neuroinflammatory conditions. Examples of neuroinflammatory conditions that can be treated by the compositions and methods described herein include, but are not limited to, multiple sclerosis (MS), acute trauma (e.g., head injury or spinal cord injury), Alzheimer's disease, amyotrophic lateral sclerosis (ALS, also known as Lou Gehrig's disease), acute disseminated encephalomyelitis, Bell's palsy, diffuse sclerosis, neurosarcoidosis, CNS complications of collagen vascular diseases (including Sjogren's disease, polyarteritis nodosa, systemic lupus erythematosus, Wegener's granulomatosis), takayasu arteritis, temporal/giant cell arteritis, tolosa-hunt syndrome, primary CNS angiitis, transverse myelitis, Susac's syndrome, PANDAS (pediatric autoimmune neuropsychiatric disorders associated with streptococcal infections), sydenham's chorea, adrenomyeloneuropathy, Guillain-Barre syndrome, chronic inflammatory demyelinating polyneuropathy, polymyositis, neurobehcet's disease, paraneoplastic syndromes, limbic encephalitis, Lambert-Eaton myasthenic syndrome and myasthenia gravis. Additional neuroinflammatory conditions are within the scope of one of skill in the art.

In some embodiments, the therapeutic compositions described herein are administered to a subject diagnosed or having symptoms of one of the above named neuroinflammatory conditions. In some embodiments, the compositions reduce or eliminate symptoms of the neuroinflammatory conditions. In some embodiments, the compositions accelerate or enhance recovery of active disease. In some embodiments, compositions are administered during episodes of acute or active disease symptoms and therapy is reduced or discontinued when disease symptoms are reduced or eliminated.

In some embodiments, compositions are given to a subject diagnosed with a neuroinflammatory condition but not exhibiting active disease symptoms (e.g., during the remission phase of a relapsing disease) in order to prevent future symptoms. For example, in some embodiments, subjects are administered the described therapeutic compositions as a long term maintenance therapy (e.g., for the remainder of their lives).

In some embodiments, G-CSF and/or G-CSFR targeted therapeutics are administered in combination with existing therapeutics. In the case of MS, such therapies include, but are not limited to, interferonβ and glatiramer acetate. Treatments for other neuroinflammatory conditions are known to those of ordinary skill in the art and include, but are not limited to, anti-inflammatory agents (e.g., non-steroidal anti-inflammatory agents and steroids), etc.

B. Pharmaceutical Compositions

The present invention further provides pharmaceutical compositions (e.g., comprising pharmaceutical agents that modulate the expression or activity of G-CSF, G-CSFR or a G-CSFR signaling molecule). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC50s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every week, month, etc.

### III. Drug Screening Applications

In some embodiments, the present invention provides drug screening assays (e.g., to screen for drugs useful in inhibiting G-CSF or G-CSFR function). For example, in some embodiments, the present invention provides methods of screening for compounds that alter (e.g., decrease) the expression of G-CSF, G-CSFR or related signaling molecules. The compounds or agents may interfere with transcription, by interacting, for example, with the promoter region. The compounds or agents may interfere with mRNA produced from G-CSF or G-CSFR (e.g., by RNA interference, antisense technologies, etc.). The compounds or agents may interfere with pathways that are upstream or downstream of the biological activity of G-CSF. In some embodiments, candidate compounds are antisense or interfering RNA agents (e.g., oligonucleotides) directed against G-CSF or G-CSFR. In other embodiments, candidate compounds are antibodies or small molecules that specifically bind to a G-CSF or G-CSFR regulator or expression product and inhibit its biological function. In some embodiments, the drug screening methods described herein identify compounds that block G-CSF binding to binding partners (e.g., those identified using the methods described herein). In some embodiments, the drug screening methods described herein identify compounds that block G-CSFR binding to binding partners (e.g., those identified using the methods described herein).

In one screening method, candidate compounds are evaluated for their ability to alter G-CSF or G-CSFR expression by contacting a compound with a cell expressing G-CSF or G-CSFR and then assaying for the effect of the candidate compounds on expression. In some embodiments, the effect of candidate compounds on expression of a G-CSF gene is assayed for by detecting the level of G-CSF mRNA expressed by the cell. In some embodiments, the effect of candidate compounds on expression of a G-CSFR gene is assayed for by detecting the level of G-CSFR mRNA expressed by the cell. mRNA expression can be detected by any suitable method. In other embodiments, the effect of candidate compounds on expression of G-CSF or G-CSFR genes is assayed by measuring the level of polypeptide encoded by the those genes. The level of polypeptide expressed can be measured using any suitable method, including but not limited to, those disclosed herein.

Specifically, the present invention provides screening methods for identifying modulators, i.e., candidate or test compounds or agents (e.g., proteins, peptides, peptidomimetics, peptoids, small molecules or other drugs) which bind to G-CSF, or have an inhibitory (or stimulatory) effect on, for example, G-CSF expression or G-CSF activity. The present invention also provides screening methods for identifying modulators, i.e., candidate or test compounds or agents (e.g., proteins, peptides, peptidomimetics, peptoids, small molecules or other drugs) which bind to G-CSFR, or have an inhibitory (or stimulatory) effect on, for example, G-CSFR expression or G-CSFR activity. Compounds thus identified can be used to modulate the activity of target gene products either directly or indirectly in a therapeutic protocol, to elaborate the biological function of the target gene product, or to identify compounds that disrupt normal target gene interactions. Compounds that inhibit the activity or expression of G-CSFs or G-CSFRs are useful in the treatment of neuroinflammatory disease (e.g., MS).

In one embodiment, the invention provides assays for screening candidate or test compounds that bind to or modulate the activity of G-CSF protein or polypeptide or a biologically active portion thereof. In another embodiment, the invention provides assays for screening candidate or test compounds that bind to or modulate the activity of G-CSFR protein or polypeptide or a biologically active portion thereof.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Natl. Acad. Sci. USA 89:18651869 [1992]) or on phage (Scott and Smith, Science 249:386-390 [1990]; Devlin Science 249:404-406 [1990]; Cwirla et al., Proc. Natl. Acad. Sci. 87:6378-6382 [1990]; Felici, J. Mol. Biol. 222:301 [1991]).

In one embodiment, an assay is a cell-based assay in which a cell that expresses a G-CSF or G-CSFR mRNA or protein or biologically active portion thereof is contacted with a test compound, and the ability of the test compound to the modulate G-CSF's or G-CSFR's activity is determined. Determining the ability of the test compound to modulate G-CSF or G-CSFR activity can be accomplished by monitoring, for example, changes in binding affinity, destruction of mRNA, or the like.

The ability of the test compound to modulate G-CSFR binding to a compound, e.g., a G-CSFR ligand, can also be evaluated. This can be accomplished, for example, by coupling the compound, e.g., the ligand, with a radioisotope or enzymatic label such that binding of the compound, e.g., the ligand, to a G-CSFR can be determined by detecting the labeled compound, e.g., ligand, in a complex.

The ability of the test compound to modulate G-CSF binding to a compound, e.g., a G-CSF receptor, can also be evaluated. This can be accomplished, for example, by coupling the compound, e.g., the receptor, with a radioisotope or enzymatic label such that binding of the compound, e.g., the receptor to a G-CSF can be determined by detecting the labeled compound, e.g., receptor, in a complex.

Alternatively, the G-CSFR is coupled with a radioisotope or enzymatic label to monitor the ability of a test compound to modulate G-CSFR binding to a G-CSFR ligand in a complex. For example, compounds (e.g., substrates) can be labeled with ¹²⁵I, ³⁵S ¹⁴C or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

Alternatively, the G-CSF is coupled with a radioisotope or enzymatic label to monitor the ability of a test compound to modulate G-CSF binding to a G-CSF receptor in a complex. For example, compounds (e.g., substrates) can be labeled with ¹²⁵I, ³⁵S ¹⁴C or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound to interact with a test protein with or without the labeling of any of the interactants can be evaluated. For example, a microphysiorneter can be used to detect the interaction of a compound with a test protein without the labeling of either the compound or test protein (McConnell et al. Science 257:1906-1912 [1992]). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and, for example, G-CSF or G-CSFR.

In yet another embodiment, a cell-free assay is provided in which a G-CSF or G-CSFR protein or biologically active portion thereof is contacted with a test compound and the ability of the test compound to bind to the G-CSF or G-CSFR protein, mRNA, or biologically active portion thereof is evaluated. Preferred biologically active portions of the G-CSF or G-CSFR proteins or mRNA to be used in assays of the present invention include fragments that participate in interactions with substrates or other proteins, e.g., fragments with high surface probability scores.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FRET) (see, for example, Lakowicz et al., U.S. Pat. No. 5,631,169; Stavrianopoulos et al., U.S. Pat. No. 4,968,103; each of which is herein incorporated by reference). A fluorophore label is selected such that a first donor molecule's emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label should be maximal. A FRET binding event can be conveniently measured through standard fluorometric detection means well known in the art (e.g., using a fluorimeter).

In another embodiment, determining the ability of the G-CSF or G-CSFR protein or mRNA to bind to a target molecule can be accomplished using real-time Biomolecular Interaction Analysis (BIA) (see, e.g., Sjolander and Urbaniczky, Anal. Chem. 63:2338-2345 [1991] and Szabo et al. Curr. Opin. Struct. Biol. 5:699-705 [1995]). “Surface plasmon resonance” or “BIA” detects biospecific interactions in real time, without labeling any of the interactants (e.g., BlAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize G-CSFs or G-CSFRs, an anti-G-CSF or G-CSFR antibody or its target molecule to facilitate separation of complexed from non-complexed forms of one or both of the proteins, as well as to accommodate automation of the assay. Binding of a test compound to a G-CSF or G-CSFR protein, or interaction of a G-CSF or G-CSFR protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase-G-CSF or G-CSFR fusion proteins or glutathione-S-transferase/target fusion proteins can be adsorbed onto glutathione Sepharose beads (Sigma Chemical, St. Louis, Mo.) or glutathione-derivatized microtiter plates, which are then combined with the test compound or the test compound and either the non-adsorbed target protein or G-CSF or G-CSFR protein, and the mixture incubated under conditions conducive for complex formation (e.g., at physiological conditions for salt and pH). Following incubation, the beads or microtiter plate wells are washed to remove any unbound components, the matrix immobilized in the case of beads, complex determined either directly or indirectly, for example, as described above.

Alternatively, the complexes can be dissociated from the matrix, and the level of G-CSF or G-CSFR binding or activity determined using standard techniques. Other techniques for immobilizing either G-CSF or G-CSFR protein or a target molecule on matrices include using conjugation of biotin and streptavidin. Biotinylated G-CSF or G-CSFR protein or target molecules can be prepared from biotin-NHS(N-hydroxy-succinimide) using techniques known in the art (e.g., biotinylation kit, Pierce Chemicals, Rockford, EL), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-IgG antibody).

This assay is performed utilizing antibodies reactive with G-CSF protein or other molecules but which do not interfere with binding of the G-CSFs protein to its target molecule. Such antibodies can be derivatized to the wells of the plate, and unbound target or G-CSF or G-CSFR protein trapped in the wells by antibody conjugation. Methods for detecting such complexes, in addition to those described above for the GST-immobilized complexes, include immunodetection of complexes using antibodies reactive with the G-CSF or G-CSFR protein or target molecule, as well as enzyme-linked assays which rely on detecting an enzymatic activity associated with the G-CSF or G-CSFR protein or target molecule.

Alternatively, cell free assays can be conducted in a liquid phase. In such an assay, the reaction products are separated from unreacted components, by any of a number of standard techniques, including, but not limited to: differential centrifugation (see, for example, Rivas and Minton, Trends Biochem Sci 18:284-7 [1993]); chromatography (gel filtration chromatography, ion-exchange chromatography); electrophoresis (see, e.g., Ausubel et al., eds. Current Protocols in Molecular Biology 1999, J. Wiley: New York.); and immunoprecipitation (see, for example, Ausubel et al., eds. Current Protocols in Molecular Biology 1999, J. Wiley: New York). Such resins and chromatographic techniques are known to one skilled in the art (See e.g., Heegaard J. Mol. Recognit. 11:141-8 [1998]; Hageand Tweed J. Chromatogr. Biomed. Sci. Appl 699:499-525 [1997]). Further, fluorescence energy transfer may also be conveniently utilized, as described herein, to detect binding without further purification of the complex from solution.

The assay can include contacting the G-CSF or G-CSFR protein, mRNA, or biologically active portion thereof with a known compound that binds the G-CSF or G-CSFR to form an assay mixture, contacting the assay mixture with a test compound, and determining the ability of the test compound to interact with a G-CSF or G-CSFR protein or mRNA, wherein determining the ability of the test compound to interact with a G-CSF or G-CSFR protein or mRNA includes determining the ability of the test compound to preferentially bind to G-CSF or G-CSFR or biologically active portion thereof, or to modulate the activity of a target molecule, as compared to the known compound.

To the extent that G-CSF or G-CSFR can, in vivo, interact with one or more cellular or extracellular macromolecules, such as proteins, inhibitors of such an interaction are useful. A homogeneous assay can be used can be used to identify inhibitors.

For example, a preformed complex of the target gene product and the interactive cellular or extracellular binding partner product is prepared such that either the target gene products or their binding partners are labeled, but the signal generated by the label is quenched due to complex formation (see, e.g., U.S. Pat. No. 4,109,496, herein incorporated by reference, which utilizes this approach for immunoassays). The addition of a test substance that competes with and displaces one of the species from the preformed complex will result in the generation of a signal above background. In this way, test substances that disrupt target gene product-binding partner interaction can be identified. Alternatively, G-CSFs or G-CSFRs protein can be used as a “bait protein” in a two-hybrid assay or three-hybrid assay (see, e.g., U.S. Pat. No. 5,283,317; Zervos et al., Cell 72:223-232 [1993]; Madura et al., J. Biol. Chem. 268.12046-12054 [1993]; Bartel et al., Biotechniques 14:920-924 [1993]; Iwabuchi et al., Oncogene 8:1693-1696 [1993]; and Brent WO 94/10300; each of which is herein incorporated by reference), to identify other proteins, that bind to or interact with G-CSFs or G-CSFRs (“G-CSF-binding proteins”, “G-CSFR-binding proteins, or “G-CSF-bp” or “G-CSFR-bp”) and are involved in G-CSF/G-CSFR activity. Such G-CSF-bps or G-CSFR-bps can be activators or inhibitors of signals by the G-CSF proteins or targets as, for example, downstream elements of a G-CSFs-mediated signaling pathway.

Modulators of G-CSFs or G-CSFRs expression can also be identified. For example, a cell or cell free mixture is contacted with a candidate compound and the expression of G-CSF or G-CSFR mRNA or protein evaluated relative to the level of expression of G-CSF or G-CSFR mRNA or protein in the absence of the candidate compound. When expression of G-CSF or G-CSFR mRNA or protein is greater in the presence of the candidate compound than in its absence, the candidate compound is identified as a stimulator of G-CSF or G-CSFR mRNA or protein expression. Alternatively, when expression of G-CSF or G-CSFR mRNA or protein is less (i.e., statistically significantly less) in the presence of the candidate compound than in its absence, the candidate compound is identified as an inhibitor of G-CSF or G-CSFR mRNA or protein expression. The level of G-CSFs or G-CSFR mRNA or protein expression can be determined by methods described herein for detecting G-CSFs or G-CSFRs mRNA or protein.

A modulating agent can be identified using a cell-based or a cell free assay, and the ability of the agent to modulate the activity of a G-CSFs or G-CSFRs protein can be confirmed in vivo, e.g., in an animal such as an animal model for a disease (e.g., an animal with neuroinflammtory disease such as MS).

This invention further pertains to novel agents identified by the above-described screening assays (See e.g., above description of therapeutic agents). Accordingly, it is within the scope of this invention to further use an agent identified as described herein (e.g., a G-CSF modulating agent, an antisense G-CSF nucleic acid molecule, a siRNA molecule, a G-CSF specific antibody, G-CSFR modulating agent, an antisense G-CSFR nucleic acid molecule, a siRNA molecule, a G-CSFR specific antibody, or a G-CSFR-binding partner) in an appropriate animal model (such as those described herein) to determine the efficacy, toxicity, side effects, or mechanism of action, of treatment with such an agent. Furthermore, novel agents identified by the above-described screening assays can be, e.g., used for treatments as described herein.

### IV. Antibodies

The present invention provides isolated antibodies. In some embodiments, the present invention provides monoclonal antibodies that specifically bind to an isolated polypeptide comprised of at least five amino acid residues of G-CSF or G-CSFR. These antibodies find use in the therapeutic and drug screening methods described herein.

An antibody against a protein of the present invention may be any monoclonal or polyclonal antibody, as long as it can recognize the protein. Antibodies can be produced by using a protein of the present invention as the antigen according to a conventional antibody or antiserum preparation process.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant may be administered. Normally, the protein is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 [1975]). As a fusion promoter, for example, polyethylene glycol (PEG) or Sendai virus (HVJ), preferably PEG is used.

Examples of myeloma cells include NS-1, P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against a tumor antigen or autoantibody of the present invention). For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO₂ gas. The antibody titer of the supernatant of a hybridoma culture can be measured according to the same manner as described above with respect to the antibody titer of the anti-protein in the antiserum.

Separation and purification of a monoclonal antibody (e.g., against a G-CSF or G-CSFR of the present invention) can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared and an animal is immunized by the complex according to the same manner as that described with respect to the above monoclonal antibody preparation. A material containing the antibody against is recovered from the immunized animal and the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to an hapten in a weight ratio of about 0.1 part to about 20 parts, preferably, about 1 part to about 5 parts per 1 part of the hapten.

In addition, various condensing agents can be used for coupling of a hapten and a carrier. For example, glutaraldehyde, carbodiimide, maleimide activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times.

The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

The protein used herein as the immunogen is not limited to any particular type of immunogen. For example, a G-CSF or G-CSFR of the present invention (further including a gene having a nucleotide sequence partly altered) can be used as the immunogen. Further, fragments of the protein may be used. Fragments may be obtained by any methods including, but not limited to expressing a fragment of the gene, enzymatic processing of the protein, chemical synthesis, and the like.

### EXPERIMENTAL

The following examples are provided in order to demonstrate and further illustrate certain preferred embodiments and aspects of the present invention and are not to be construed as limiting the scope thereof.

### Example 1

### Blood Derived Myeloid Cells Comprise a Major Component of EAE Infiltrates

Although myelin-specific CD4+ T cells initiate CNS inflammation during EAE, CD45hiCD11b+ myeloid cells comprise a major constituent of established white matter infiltrates. Over 50% of spinal cord mononuclear cells harvested from mice at peak disease have this phenotype (FIG. 1A). Among CNS CD11b+ cells there is a distinct subset that expresses the cell surface phenotype CD11c+CD8α−, indicative of myeloid dendritic cells (FIG. 1B).

Lymph node and splenic myeloid dendritic cells are potent APCs that can induce uncommitted CD4+ T cells to differentiate along Th1 or Th17 lineages. It was contemplated that CD11c+CD8α− cells play similar roles in the inflamed CNS. In fact, CD11c+ cells form clusters with CD4+ cells within EAE lesions (FIG. 1C). Furthermore, they are highly efficient at stimulating both naïve and primed myelin-specific T cells to proliferate and secrete effector cytokines such as IFNγ and IL-17 ex vivo (FIG. 2). In addition, they secrete IL-23, a cytokine that plays a critical role in EAE pathogenesis, during coculture with myelin-specific T cells (FIG. 3). While CD45hiCD11b+CD11c− macrophages are not as efficient at activating naïve T cells, they readily stimulate memory myelin-reactive T cells to secrete IFNγ (FIG. 2).

In independent experiments the origin of the CNS myeloid cells that accumulate in EAE lesions was investigated. Previously published studies indicated that adult microglia could differentiate into CD11c+ cells with morphological and functional characteristics of dendritic cells following culture with proinflammatory growth factors in vitro. However, experiments with bone marrow chimeric mice indicate that virtually all CD45hiCD11b+ cells in the CNS of mice with EAE, including CD11c+ cells, originate from radiosensitive donor hematopoietic cells rather than radioresistant CNS resident cells, such as microglia (FIG. 4).

### Myelopoiesis is Accelerated During EAE

As shown in FIG. 4, the vast majority of APCs in EAE lesions infiltrate from the periphery. However, relatively little is known about how myeloid cells are regulated in the bone marrow, secondary lymphoid tissues and circulation during EAE. Following extravasation from the blood stream, monocytes have a relatively short life span, surviving only a matter of 2-4 days (Whitelaw, Blood 28, 455-464 (1966)). The number of macrophages and dendritic cells in the CNS contracts during remissions and rebounds during exacerbations of EAE (Begolka et al, supra; Jee et al., supra). Collectively, these observations indicate that CNS myeloid cells are periodically replenished with peripheral precursors during the course of relapsing autoimmune demyelinating disease. Therefore, it was concluded that myeloid progenitor cells must be released from the bone marrow at a heightened rate prior to, or in concert with, clinical disease activity. To investigate that hypothesis, myelin-immunized SJL mice were bled at serial time points according to clinical stage and the frequency of granulocyte-macrophage colony forming units (CFU-GM) in methylcellulose culture with GM-CSF and stem cell factor was measured. As shown in FIG. 5A, the frequency of circulating CFU-GM consistently rose shortly before the clinical onset and relapse of EAE, but fell during remission.

To identify the cellular source of CFU-GM in the circulation of myelin-immunized mice, methylcellulose cultures were set up with leukocytes sorted for cell surface profiles indicative of different maturation stages (FIG. 5B) (Sunderkotter, C. et al. J Immunol 172, 4410-4417 (2004). Relatively immature Ly6ChiCD11b+ cells, as opposed to Ly-6Cint or Ly-6Cneg cells, gave rise to CFU-GM at a high frequency (FIG. 5C). The majority of colonies contained 8-50 cells indicative of 3-5 cell divisions. Ly-6Chi blood leukocytes had bean-shaped nuclei typical of the monocyte/macrophage lineage (FIG. 5D) and uniformly expressed the macrophage colony-stimulating factor receptor, CD115 (FIG. 5E). Ly-6Chi precursors also expressed CD62L, 7/4 and F4/80, but were Ly-6G−, indicative of an inflammatory monocyte phenotype (FIG. 5E) (Geissmann et al., Immunity 19, 71-82 (2003)). Furthermore, the circulating Ly-6Chi cells have forward and side scatter characteristics that fall within a monocyte, as opposed to a granulocyte gate.

Ly6Chi CD11b+ Monocytes Accumulate in the Blood and CNS of Myelin-Immunized Mice

Ly-6Chi CD11b+ cells first began accumulating in the blood and CNS during the preclinical stage of disease (FIGS. 6A, B and D). Immediately prior to the onset of clinical disease the vast majority of Ly-6Chi CD11b+ cells in the spinal cord are CD11c− MHC Class II-/lo. However, during the symptomatic stage, the CNS Ly-6Chi CD11b+ cell population is predominantly CD11c+ and MHC Class IIint/hi (FIG. 6D, lower panels). Hence, immature monocytes that accumulate in the CNS during the preclinical stage either differentiate in situ or are replaced by a more mature subset as EAE evolves. Identical results were obtained with EAE induced by the adoptive transfer of myelin-specific T cells.

### Ly-6C+ Monocytes Traffic to the CNS and Express a Pro-Inflammatory Phenotype.

The above studies show that Ly-6Chi CD11b+ cells are mobilized into the circulation at an increased rate prior to EAE exacerbations, accumulate in the CNS during the preclinical phase of EAE, and differentiate into DC upon stimulation in vitro. It was speculated that circulating Ly-6Chi cells home to the CNS and give rise to mature myeloid populations during EAE.

Under homeostatic conditions, intravenous injection of FITC conjugated microspheres results in preferential labeling of circulating Ly6C−CD11b+ cells due to their relatively high phagocytic capacity. By contrast, the microspheres are selectively taken up by Ly6Chi cells when injected into clodronate treated mice at the time of peripheral leukocyte reconstitution (Tacke et al., J Exp Med 203, 583-597 (2006)). Host mice were injected with clodronate liposomes and FITC-conjugated microspheres 24 and 48 hours following the adoptive transfer of myelin-specific T cells, respectively. Subsequent FACS analysis confirmed that the vast majority of FITC+ peripheral blood mononuclear cells (PBMC) from the clodronate treated group were CD11b+Ly-6Chi while FITC+PBMC from control mice treated with PBS liposomes were predominantly CD11b+Ly-6Cneg (FIG. 7A).

By the time of peak EAE, a significant proportion of FITC+ cells had infiltrated the CNS of the adoptive transfer hosts that received clodronate liposomes, but not the CNS of hosts that had received PBS liposomes (FIG. 7B). Virtually all of the CNS infiltrating FITC+ cells were F4/80+Ly6Chi MHCIIhi and CD11cint or neg (FIG. 7C), implying that circulating Ly-6Chi CD11b+ cells give rise to the mature macrophages and myeloid dendritic cells that populate EAE lesions. These results were corroborated by parallel studies in which CD45.2+Ly-6Chi blood monocytes, transferred into CD45.1 congenic hosts with active EAE, developed into Ly6Chi MHCIIhi CD11cint/neg CNS mononuclear cells (FIG. 8). By comparison to Ly-6Chi cells in the blood, Ly-6C+MHCII+ cells in the CNS upregulated mRNA encoding p40 (the common subunit of IL-12 and IL-23), p19 (the IL-23 specific subunit), and IL-6 (FIG. 7D) (Oppmann et al., Immunity 13, 715-725 (2000)). Each of these genes has previously been shown to be indispensable for the induction of EAE. CNS Ly-6C+MHCII+ cells also expressed elevated levels of TNFα and iNOS (FIG. 7D). Taken together, these data demonstrate that Ly-6Chi blood monocytes are a source of mature CNS-infiltrating myeloid cells during EAE and that they acquire a pro-inflammatory genetic profile having crossed the blood brain barrier.

### Increasing the Frequency of Ly-6Chi Precursors During the Effector Phase of EAE Accelerates the Onset and Severity of Disease

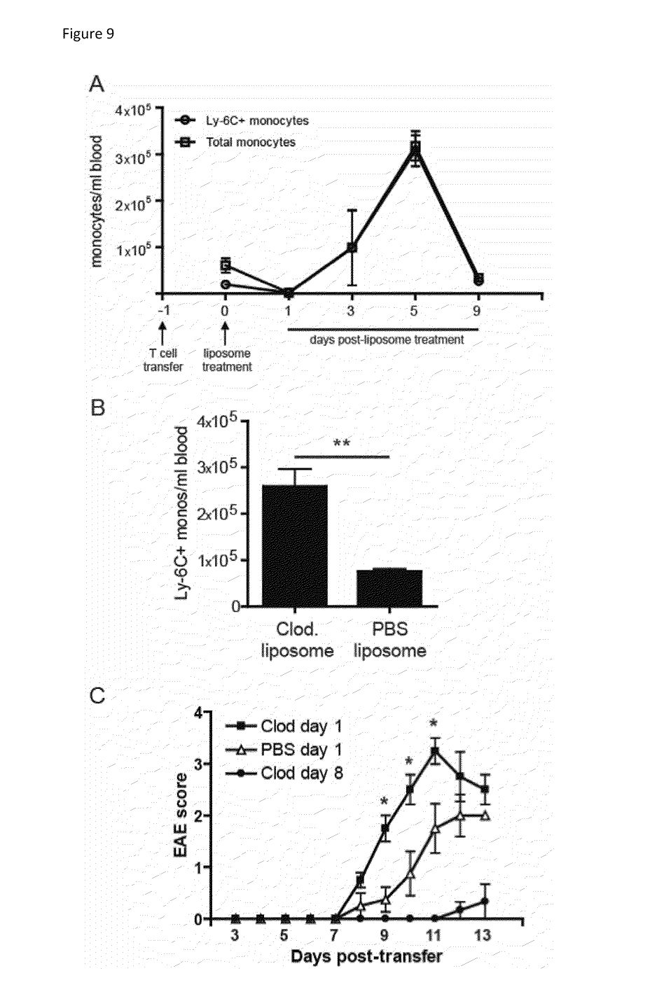

As described above, intravenous treatment of mice with clodronate liposomes results in enrichment of Ly-6C+ cells in the circulating monocyte pool but does not affect other leukocyte subsets. Ly6Chi cells comprise the vast majority of CD115+ cells in the blood for 7-10 days after the intervention (FIG. 9A). These cells are CD11b+F4/80+CD62L+Ly6G−, consistent with the cell surface profile of inflammatory monocytes (Geissmann et al., supra). Based on data showing that Ly6Chi blood cells develop into CNS Ly-6C+MHCII+ cells with a proinflammatory profile (FIG. 7D), it was predicted that administration of clodronate liposomes shortly following adoptive transfer would exacerbate the clinical course of EAE. Mice were injected with clodronate or PBS liposomes 18 hours after receiving encephalitogenic CD4+ T cells. As shown in FIG. 9A, clodronate liposome treatment initially depleted all circulating CD115+ cells. Newly mobilized monocytes, that were skewed towards the immature Ly-6C+ subset, reconstituted the blood within 2 days. Consequently, from day 3 onward, clodronate liposome treated mice had a significantly higher number of Ly-6C+ cells/ml blood than PBS liposome treated controls (FIG. 9B). As expected, administration of clodronate liposomes shortly after adoptive transfer resulted in an accelerated and more severe clinical course (FIG. 9C). By contrast, repetitive treatment with clodronate liposomes beginning eight days after T cell transfer (a regimen that wholly depletes all monocytes from the circulation throughout the effector stage) suppressed EAE and delayed its onset (FIG. 9C).

### The Role of GM-CSF in Regulating Peripheral Myeloid Cells During EAE

GM-CSF deficient (−/−) mice and wildtype mice treated with neutralizing antibodies to GM-CSF are resistant to EAE (McQualter et al., J Exp Med 194, 873-882 (2001)). MOG immunized GMCSF−/− mice mount reduced IL-2 and IFNγ recall responses, indicating that the cytokine plays a role in autoreactive CD4+ T cell priming and/or Th differentiation, likely via indirect effects on antigen presenting cells (Wada et al., Proc Natl Acad Sci USA 94, 12557-12561 (1997)). However, GM-CSF has pleiotrophic functions, indicating that it may act at multiple steps in autoimmune pathogenesis (Hamilton, Trends Immunol 23, 403-408 (2002)). GM-CSF is an important growth factor for the expansion and differentiation of myeloid cells. It induces bone marrow progenitors to develop into myeloid dendritic cells in vitro (Inaba et al., J Exp Med 176, 1693-1702 (1992)). GM-CSF also can enhance the mobilization of myeloid cells at different stages of development from the bone marrow.

The present invention is not limited to a particular mechanism. Indeed, an understanding of the mechanism is not necessary to practice the present invention. Nonetheless, it is contemplated that one mechanism by which G-CSF could promote EAE is to accelerate the release of bone marrow Ly-6Chi precursors that ultimately differentiate into CNS infiltrating DC and macrophages. Indeed, while circulating Ly-6C+ monocytes expanded over 60 fold immediately prior to expected EAE onset in WT mice, their levels remained stable in GM-CSF−/− mice that were actively immunized in an identical fashion (FIG. 10A). Similar results were obtained when myelin-immunized WT mice were treated with neutralizing antibodies to GM-CSF (FIG. 10B). Conversely, administration of recombinant GMCSF to immunized GM-CSF−/− mice triggered Ly-6Chi monocyte mobilization and restored susceptibility to EAE (FIG. 10C).

GM-CSF Receptor Sufficient and Deficient Ly6ChiCD11b+ Cells Populate the Blood and CNS During the Preclinical Stage of EAE in Equal Proportions

GM-CSF receptor deficient mice fail to mobilize myeloid progenitor cells into the circulation and remain resistant to EAE in response to challenge with myelin antigens in CFA as well as following the adoptive transfer of myelin-specific T cells (FIG. 10). It was next investigated whether GM-CSF induces myeloid cells to migrate from the bone marrow into the circulation by a direct or indirect pathway. To do so, bone marrow chimeric mice were constructed by injecting lethally irradiated CD45.1+CD45.2+ hosts with a mixture of CD45.1+WT and CD45.2+GM-CSFR−/− bone marrow cells. Flow cytometry revealed that, 6 weeks following reconstitution, approximately 50% of circulating monocytes cells were CD45.1+CD45.2− (indicating derivation from WT donors) and 50% were CD45.1-CD45.2+ (indicating derivation from GM-CSFR−/− donors). This was not surprising since GM-CSF−/− and GM-CSFR−/− have grossly normal numbers of peripheral blood mononuclear cells and myeloid cells in secondary lymphoid tissues during homeostasis (Dranoff et al., Science 264, 713-716 (1994); Robb et al., Proc Natl Acad Sci USA 92, 9565-9569 (1995); Stanley et al., Proc Natl Acad Sci USA 91, 5592-5596 (1994); Vremec et al., Eur J Immunol 27, 40-44 (1997)). Ten days following the immunization of chimeric mice with myelin antigens (two days before the expected onset of clinical EAE), comparable numbers of Ly6ChiCD11b+ cells in the blood and CNS were derived from WT and GM-CSFR−/− donors (FIG. 11). This indicates that GM-CSF mobilizes bone marrow myeloid cells by an indirect mechanism. The data in FIG. 11 also demonstrate that direct GM-CSF stimulation is not required for the passage of Ly6ChiCD11b+ cells across the blood-brain-barrier or for their retention in the CNS immediately prior to EAE exacerbations.

### CXCR2 and EAE

IL-23 induces IL-17 that, in turn, induces GM-CSF and ELR+CXC chemokines (Ouyang et al., Immunity 28, 454-467 (2008)). ELR+CXC chemokines trigger the rapid mobilization of CFU-GM from the bone marrow (Pelus et al., Crit Rev Oncol Hematol 43, 257-275 (2002)). They can also synergize with GM-CSF to promote continual export of the progenitor cells into the circulation. It is currently believed that these chemokines stimulate neutrophils in the bone marrow to secrete proteases, such as matrix metalloproteinase (Epstein et al., J Neurol Sci 61, 341-348 (1983)), that digest the connective tissue matrix and degrade chemotactic factors, thereby expediting the release of progenitor cells (Christopher et al., Blood (2009); Lapidot et al., Exp Hematol 30, 973-981 (2002); Levesque et al., Blood 98, 1289-1297 (2001); Pelus et al., Blood 103, 110-119 (2004)). CXCR2 is the only receptor for ELR+CXC chemokines in mouse. Furthermore, it appears to be the dominant receptor for the effects of ELR+CXC chemokines on bone marrow cells across species. It was found that CXCR2 deficient mice are resistant to EAE (FIG. 12).

### Construction of G-CSF Receptor-Fc and CXCR2-Fc Fusion Proteins

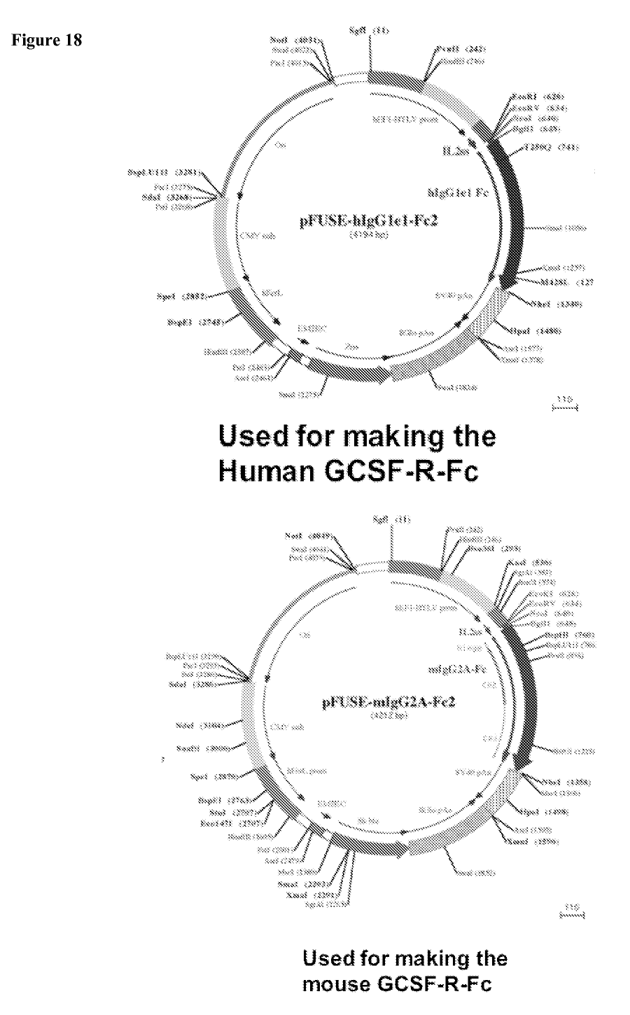

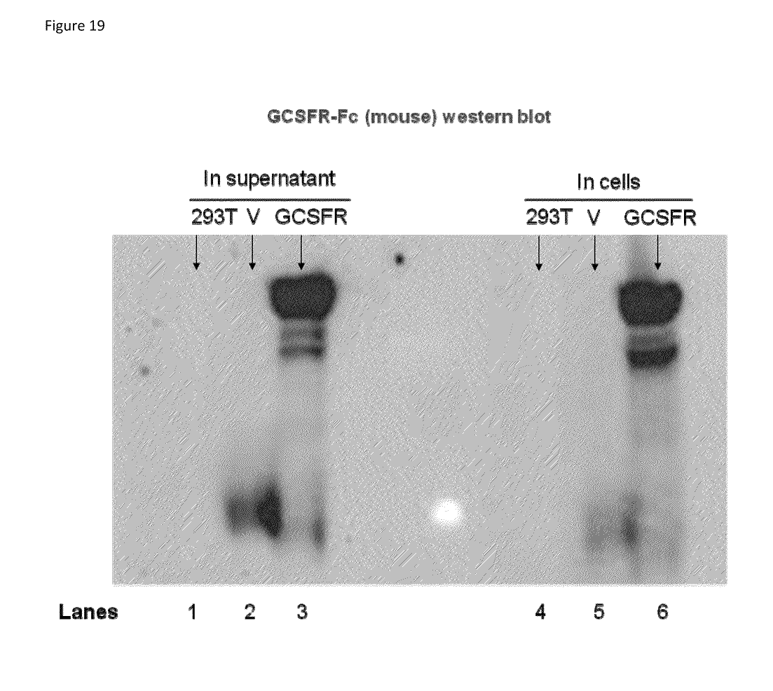

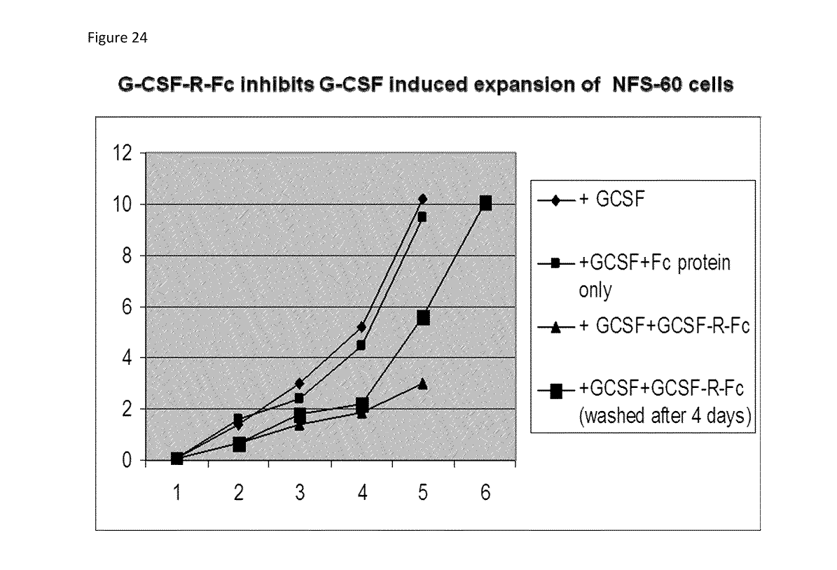

In order to obtain reagents for blocking G-CSF and ELR+CXC chemokines in vivo, Fc-fusion proteins were constructed using the plasmid pFUSE-mIgG2A-Fc2 (InvivoGen). This plasmid contains genes encoding the IL-2 signal sequence (IL-2ss, to facilitate secretion of the Fc-Fusion proteins), the Fc region of mouse IgG2A heavy chain (CH2 and CH3 domains and the hinge region), and a multiple cloning site (FIG. 18). The extracellular domain of GCSF receptor (G-CSFR) was PCR amplified from cDNA derived from NSF-60 cells using primers with the following sequences: 5′ ATC GGA TCC TGT GGA CAC ATC GAG ATT TCA 3′ (forward); 5′ ATC GGA TCC GTC AGA TGG ATC ATG GGT CCT CAG G 3′ (reverse). The sequence of the amplified product was confirmed by the University of Michigan Sequencing Core Facility. Amplified G-CSFR fragments were then inserted in the pFUSE-mIgG2A-Fc2 plasmid using EcoRI/NcoI restriction sites located between the IL-2ss and Fc genes. Recombined plamids were transfected into human embryonic kidney (HEK) 293T cells and selected with Zeocin. The presence of fusion protein in the conditioned media (supernatant) and cell lysates (cells) of transfectants was confirmed by Western blot analysis (FIG. 19). A large quantity of fusion protein was purified from supernatants by protein A affinity chromotography. The bioactivity of purified fusion protein was assessed by its ability to inhibit G-CSF driven proliferation of NSF-60 cells (FIG. 24). G-CSFR-Fc blocked mobilization of neutrophils into the circulation when administered in combination with recombinant G-CSF or cyclophosphamide in vivo. Furthermore, injection of myelin immunized mice with G-CSFR-Fc shortly after the clinical onset of EAE, suppressed disease throughout the duration of treatment (FIG. 20). Mice began exhibiting increased signs of disability two days after the final dose was given.

### Example 2

### Investigation of the Cytokine Pathways and Molecular Mechanisms Underlying the Expansion and Mobilization of Bone Marrow Myeloid Cells that Occur Prior to EAE Exacerbations

### G-CSF and ELR+CXC Chemokines Act Downstream of GM-CSF to Promote Myeloid Cell Mobilization Prior to Exacerbations of EAE.

As demonstrated (FIG. 5A), CD11b+CD62L+Ly6Chi monocytes with colony forming potential are mobilized into the bloodstream immediately preceding clinical EAE onset and relapse. Adoptive transfer experiments indicate that these newly exported cells ultimately give rise to CNS infiltrating monocytes, macrophages and dendritic cells at peak disease (FIGS. 7, 8). Several lines of evidence support a pathogenic role of Ly6Chi monocytes in autoimmune demyelination. First, enrichment of Ly6Chi monocytes in the circulation is associated with an earlier onset and increased severity of clinical EAE (FIG. 9). Conversely, repetitive depletion of blood monocytes throughout the effector phase of EAE ameliorates neuroinflammation and clinical deficits. Furthermore, the resistance of CD62L deficient mice to EAE is reversed by reconstitution with wildtype monocytes (Grewal, et al. Immunity 14: 291-302 (2002)).

Circulating CD11b+CD62L+Ly6Chi monocytes and CFU-GM do not increase in frequency following active immunization of GM-CSF deficient (GMCSF−/−) and GM-CSF receptor deficient (GM-CSFR−/−), as opposed to WT, mice with myelin peptides (FIG. 10A). Similarly, treatment of actively immunized WT mice with anti-GM-CSF neutralizing antibodies prevents the release of myeloid precursors from the bone marrow, whereas injection of GM-CSF−./− mice with recombinant GM-CSF has the opposite effect (FIG. 10 B,C). GM-CSF is a plieotrophic cytokine that acts at multiple steps in autoimmune pathogenesis, including the priming of effector Th responses (McQualter et al., J Exp Med 194, 873-882 (2001)). However, GM-CSF signaling is critical at a stage beyond effector T cell priming since GM-CSFR−/− mice injected with normally encephalitogenic T cells do not mobilize myeloid cells or succumb to EAE. G-CSF acts synergistically with GM-CSF and ELR+CXC chemokines to promote the mobilization of inflammatory monocytes from the bone marrow (Pelus, et al. Exp Hematol. 34, 1010-1020 (2006)). The present invention is not limited to a particular mechanism. Indeed, an understanding of the mechanism is not necessary to practice the present invention. Nonetheless, it is contemplated that given the importance of CNS infiltration by macrophages and dendritic cells for the development of relapsing neurological deficits (Greter et al., Nat Med 11, 328-334 (2005); Tran et al., J Immunol 161, 3767-3775 (1998); Huang et al., J Exp Med 193, 713-726 (2001)), G-CSF driven myeloid cell mobilization is one of the mechanisms by which this cytokine promotes autoimmune disease, including EAE.

The role of G-CSF and ELR+CXC chemokines (in synergy with, or downstream of, GM-CSF) in the accelerated myeloid moblilization that occurs immediately prior to EAE exacerbations is examined. It is contemplated that GM-CSF, secreted and induced by autoreactive T cells, stimulates bone marrow stromal cells to secrete G-CSF and CXCL1/2. G-CSF and the ELR+CXC chemokines, in turn, activate bone marrow resident neutrophils to secrete proteases that degrade chemokines and adhesion molecules critical for the sequestration of hematopoietic precursor cells in intramedullary niches. It is contemplated that interruption of any step in this pathway (for example, by neutralization of G-CSF) will prevent release of bone marrow myeloid cells, ultimately leading to exhaustion of peripheral monocyte pools that give rise to CNS-infiltrating macrophages and dendritic cells necessary to support relapsing and chronic neuroinflammation. Such interventions are of therapeutic benefit in EAE.

In addition to inducing G-CSF and CXCL1/2, GM-CSF could act cooperatively with those factors to promote release of bone marrow cells into the circulation. In an experimental model of Listeria infection, G-CSF and GM-CSF act cooperatively to incite macrophage infiltration at the site of infection and drive protective immunity (Zhan et al., Blood 91, 863-869 (1998)). In both human and murine studies, administration of GM-CSF and G-CSF, either consecutively, or in combination, is more effective than either factor alone in mobilizing myeloid cells following chemotherapeutic ablation. Synergism has been observed between recombinant CXCL2 and G-CSF in a similar manner (Wang et al., J Leukoc Biol 62, 503-509 (1997)).

It is contemplated that levels of GM-CSF, G-CSF and ELR+CXC chemokines will rise in the sera and bone marrow extracellular fluid of actively immunized mice immediately before the onset and relapse of clinical EAE and during clinical progression, and fall during remissions. This pattern of growth factor/chemokine expression will mirror fluctuations in the frequency of circulating Ly6Chi monocytes and CFU-GM during the course of relapsing disease.

It is also contemplated that GM-CSF neutralization will inhibit upregulation of G-CSF and ELR+CXC chemokines in the sera and bone marrow extracellular fluid of actively immunized mice.

It is further contemplated that administration of G-CSFR-Fc and CXCR2-Fc fusion proteins will block enhanced myeloid cell mobilization and suppress clinical EAE.

The G-CSFR fusion protein has been purified from the condition media of transformed 293T cells and its biological activity confirmed both in vitro and in vivo (FIGS. 19 and 24). It is capable of suppressing EAE exacerbations (FIGS. 20-22). In addition to contemplated uses of CXCR2-Fc chimeric protein, alternative strategies include use of an anti-CXCR2 antiserum to suppress myeloid cell mobilization during EAE.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

We claim:

1. A pharmaceutical composition comprising at least one pharmaceutical agent that inhibits at least one activity of a recombinant granulocyte colony stimulating factor (G-CSF) or a recombinant granulocyte colony stimulating factor receptor (G-CSFR) protein.

2. The composition of claim 1, wherein said pharmaceutical agent comprises an FC-fusion protein.

3. The composition of claim 1, wherein said pharmaceutical agent is selected from the group consisting of an antibody that binds to said G-CSF, an antibody that binds to said G-CSFR, an siRNA that inhibits the expression of said G-CSF, an siRNA that inhibits the expression of said G-CSFR, an antisense oligonucleotide that inhibits the expression of said G-CSF, an antisense oligonucleotide that inhibits the expression of said G-CSFR and a small molecule.

4. The composition of claim 1, wherein said composition reduces or eliminates symptoms of a neuroinflammatory condition.

5. The composition of claim 1, wherein said composition prevents relapses of a neuroinflammatory condition.

6. The composition of claim 5, wherein said neuroinflammatory condition is multiple sclerosis.

7. A method of treating a neuroinflammatory condition, comprising:
administering a composition that inhibits at least one activity of a G-CSF or G-CSFR protein under conditions such that symptoms of said neuroinflammatory condition are reduced or eliminated.

8. The method of claim 7, wherein said neuroinflammatory condition is multiple sclerosis.

9. The method of claim 7, wherein said composition comprises an FC-fusion protein.

10. The method of claim 7, wherein said composition is selected from the group consisting of an antibody that binds to said G-CSF, an antibody that binds to said G-CSFR, an siRNA that inhibits the expression of said G-CSF, an siRNA that inhibits the expression of said G-CSFR, an antisense oligonucleotide that inhibits the expression of said G-CSF, an antisense oligonucleotide that inhibits the expression of said G-CSFR and a small molecule.

11. The method of claim 7, wherein said composition accelerates recovery of said subject from symptoms of said neuroinflammatory condition.

12. The method of claim 7, wherein said composition prevents relapses of symptoms of said neuroinflammatory condition.
